(19) United States
(12) Patent Application Publication
(10) Pub. No.: US 2024/0016787 A1
(43) Pub. Date: Jan. 18, 2024

(54) METHODS FOR TREATMENT OF CANCER AND PHAGOCYTOSIS-DEFICIENCY RELATED DISEASES

(71) Applicant: RDiscovery, LLC, Seattle, WA (US)

(72) Inventors: Taro Inaba, Tokyo (JP); Kazuhiko Nonomura, Chiba City (JP)

(73) Assignee: RDiscovery, LLC, Seattle, WA (US)

(21) Appl. No.: 18/251,443

(22) PCT Filed: Nov. 2, 2021
(86) PCT No.: PCT/US21/57698

§ 371 Date: May 2, 2023

Related U.S. Application Data
(60) Provisional application No. 63/109,111, filed on Nov. 3, 2020.
(60) Provisional application No. 63/145,681, filed on Feb. 4, 2021.

(51) Int. Cl. A61K 31/4422; A61K 31/155; A61K 38/10; A61K 31/517; A61K 31/4375; A61K 35/745; A61K 35/747; A61K 31/575; A61K 39/395; A61K 38/17; A61K 31/7068; A61K 31/337; A61K 33/243; A61K 31/282; A61K 39/00; A61P 35/00

(52) U.S. Cl. CPC A61K 31/4422; A61K 31/155; A61K 38/10; A61K 31/517; A61K 31/4375; A61K 35/745; A61K 35/747; A61K 31/575; A61K 39/3955; A61K 38/177; A61K 31/7068; A61K 31/337; A61K 33/243; A61K 31/282; A61K 39/4614; A61K 39/4615; A61P 35/00

(57) ABSTRACT

Methods for treating diseases and conditions associated with phagocytosis deficiency and methods for treating cancer with phagocytosis activating agents and mitochondrial fission inhibitors, respectively, as monotherapy or in combination with one or more additional agents, and agents, combinations of agents, and compositions for treating diseases and conditions associated with phagocytosis deficiency.

### 1. CROSS-REFERENCE TO RELATED APPLICATIONS

This application claims the priority benefit of U.S. provisional application Ser. Nos. 63/109,111, filed Nov. 3, 2020, and 63/145,681, filed Feb. 4, 2021, the contents of each of which are incorporated herein in their entireties by reference thereto.

### 2. BACKGROUND

CD47 is a broadly expressed cell surface protein that binds to signal-regulatory protein α (SIRPα) on macrophages to protect healthy cells from macrophage engulfment. The interaction between CD47 and SIRPα is called the “don't eat me” signal. CD47 is often upregulated on cancer cells, enabling the cancer cells to evade innate immune detection. Morissey and Vale, 2019, bioRxiv 752311; doi.org/10.1101/752311. By blocking the interaction between CD47 and SIRPα, for example, by an anti-CD47 or anti-SIRPα antibody, the “don't eat me” signal is weakened and the phagocytic function of macrophages and other phagocytes should be activated. Anti-CD47 and anti-SIRPα antibodies have generated promising clinical and non-clinical data in the oncology area. However, a significant number of patients do not respond to therapies targeting the CD47-SIRPα interaction (see, e.g., Advani et al., 2018, N Engl J Med. 379 (18):1711-1721).

Beyond cancer, phagocytosis deficiency is also associated with other diseases, for example, infectious diseases, neurodegenerative diseases, inflammation, and lysosomal diseases (see, e.g., Engelich et al., 2001, Clinical Infectious Diseases 33 (12):2040-2048; Andrews and Sullivan, 2003, Clin Microbiol Rev. 16 (4):597-621; Galloway et al., 2019, Front Immunol. 10:790; Kourtzelis et al., 2020, Front Immunol. 2020; 11: 553; Maderna and Godson, 2003, Biochimica et Biophysica Acta (BBA)—Molecular Basis of Disease, 1639 (3):141-151; Marques and Saftig, 2019, Journal of Cell Science 132:jcs221739; Parkinson-Lawrence et al., 2010, Physiology 25:102-115).

There remains a need for new therapies for treating cancer and phagocytosis deficiency-related diseases and conditions.

### 3. SUMMARY

Without being bound by theory, it is believed that the limited clinical and non-clinical effects of CD47-SIRPα targeting therapies are, at least in some cases, associated with a deficiency in phagocytic ability of phagocytes such as monocytes, macrophages, and microglia. It is believed that phagocytic deficiency can be due to various reasons including nutritional conditions, poor stimulation of innate immunity, polarization of phagocytes, aging-associated senescence of hematopoietic stem cells and their lineage cells, including monocytes. It is believed that when phagocytic capacity of phagocytes is suboptimal, blockade of the CD47 and SIRPα interaction may not be sufficient for phagocytes to eliminate cancer cells or induce the acquired immune system to develop an immunity against cancer cells.

It is further believed, again without being bound by theory, that enhancing phagocytic activity of phagocytes by phagocytosis activating agents can improve outcomes in non-cancer phagocytosis deficiency-related diseases and conditions, for example infectious diseases, neurodegenerative diseases, inflammation, inflammatory diseases, lysosomal diseases, and musculoskeletal degenerative diseases such as Duchenne muscular dystrophy (DMD).

Accordingly, in one aspect, the disclosure provides methods of treating phagocytosis-deficiency related diseases and conditions, for example, cancers, with an agent that activates phagocytosis, for example, a mitochondrial fission inhibitor such as cilnidipine, P110, metformin, mdivi-1, or berberine. The present disclosure is based, in part, on the discovery that the mitochondrial fission inhibitor cilnidipine is able to enhance phagocytic activity of macrophages. The phagocytosis activating agent can be administered as monotherapy or, alternatively, can be administered in combination with an additional agent or combination of agents, for example an anti-CD47 antibody and/or an anti-SIRPα antibody. In some embodiments, the phagocytosis-deficiency related disease is a cancer. In other embodiments, phagocytosis-deficiency related disease is a non-cancer disease associated with phagocytic dysfunction, for example an infectious disease, a neurodegenerative disease, an inflammatory disease, a lysosomal disease, or a musculoskeletal degenerative diseases such as DMD.

Exemplary phagocytosis activating agents are described in Section 5.1 and specific embodiments 2 to 89, infra. Exemplary additional agents and combinations of agents that can be used in combination with a phagocytosis activating agent or combination of phagocytosis activating agents are described in Section 5.2 and specific embodiments 111 to 148, infra. Exemplary diseases associated with phagocytosis deficiency are described in Section 5.4 and specific embodiments 1 and 149 to 186, infra.

In another aspect, the disclosure provides methods of treating cancer (which may or may not be associated with phagocytosis deficiency) with mitochondrial fission inhibitors such as cilnidipine, a cilnidipine derivative (e.g., as described in WO 2020/241638), P110, metformin, mdivi-1, or berberine. In some embodiments, the cancer is has a KRAS mutation. KRAS mutations can drive cancer cells to grow and spread in the body, and, without being bound by theory, it is believed that mitochondrial fission inhibitors can inhibit the proliferative signal caused by KRAS mutations in tumor cells.

The mitochondrial fission inhibitor can be administered as monotherapy for the cancer or, alternatively, administered as part of a combination therapy, for example with an anti-PD1 or anti-PD-L1 antibody. In some embodiments, a combination therapy comprises a mitochondrial fission inhibitor, e.g., cilnidipine, administered with a standard of care therapy. Exemplary mitochondrial fission inhibitors are described in Section 5.1.1 and specific embodiments 5 to 54, infra. Exemplary cancers that can be treated are described in Section 5.4.1 and specific embodiments 90 to 109, infra.

In further aspects, the disclosure provides agents and combinations of agents for use in the methods described herein. The agents and combinations of agents can be in pharmaceutical compositions. Exemplary agents, combinations of agents and pharmaceutical compositions are described in Sections 5.1 to 5.3 and specific embodiments 187 to 237, infra.

In yet further aspects, the disclosure provides pharmaceutical compositions comprising phagocytes having enhanced phagocytic activity. Phagocytes having enhanced phagocytic activity can be obtained by treating phagocytes ex-vivo with a mitochondrial fission inhibitor, for example cilnidipine. Such pharmaceutical compositions can be used in methods of treating a subject having a phagocytosis deficiency-related disease or condition, for example a cancer or other disease or condition disclosed herein. The phagocytes can be autologous to the subject to be treated or, alternatively, the phagocytes can be allogeneic to the subject to be treated. Exemplary pharmaceutical compositions comprising phagocytes having enhanced phagocytic activity, processes for their production, and mitochondrial fission inhibitors for use in preparing such pharmaceutical compositions are described in Section 5.3 and specific embodiments 238 to 255, infra.

### 4. BRIEF DESCRIPTION OF THE FIGURES

FIG. 1 shows the enhancement of phagocytic activity in macrophages exposed to DMSO vehicle or cilnidipine at a concentration of 0.3 μM, 1 μM, or 3 μM. Phagocytic ratio is the ratio of cells that engulfed the indicated number of fluorescently labeled particles (one particle or more, two particles or more, three particles or more, or four particles or more).

FIG. 2 shows mean fluorescence intensity derived from fluorescently-labeled beads taken up by macrophages exposed to DMSO vehicle or cilnidipine at a concentration of 0.3 μM, 1 μM, or 3 μM.

FIG. 3 shows body weights of animals of Example 4.

FIG. 4 shows rotarod latency results for animals of Example 4. Data are presented as mean+SEM.

FIG. 5 shows plasma LDH levels for animals of Example 4. Data are presented as mean+SEM; ### represents p<0.001 by unpaired t test vs. WT; * represents p<0.05 by unpaired t test vs. MDX-Vehicle.

FIG. 6 shows plasma CK levels for animals of Example 4. Data are presented as mean+SEM; #π# represents p<0.001 by unpaired t test vs. WT; * represents p<0.05 by unpaired t test vs. MDX-Vehicle.

FIGS. 7A-7B show mean body weight (FIG. 7A) and percent body weight change (FIG. 7B) for animals of Example 5.

FIGS. 8A-8B shows mean tumor volume (FIG. 8A) and percent inhibition of tumor volume (FIG. 8B) for animals of Example 5.

### DETAILED DESCRIPTION

In one aspect, the disclosure provides methods of treating phagocytosis-deficiency related diseases and conditions, for example, cancers, with agents that activate phagocytosis, either as monotherapy or, alternatively, in combination with an additional agent or combination of agents, for example an anti-CD47 antibody and/or an anti-SIRPα antibody. Exemplary phagocytosis activating agents are described in Section 5.1. Exemplary additional agents and combinations of agents are described in Section 5.2. Pharmaceutical compositions that can be used in the methods of the disclosure are described in Section 5.3. Exemplary diseases associated with phagocytosis deficiency are described in Section 5.4.

In another aspect, the disclosure provides methods of treating cancer (which may or may not be associated with phagocytosis deficiency) with mitochondrial fission inhibitors such as cilnidipine or a cilnidipine derivative. Exemplary mitochondrial fission inhibitors are described in Section 5.1.1. Exemplary cancers that can be treated are described in Section 5.4.1. The methods of treating cancer can in some embodiments comprise administering an amount of an agent or combination of agents effective to slow the progression of a subject's cancer (e.g., slow the rate of tumor growth).

In yet another aspect, the disclosure provides methods of treating non-cancer diseases associated with phagocytic dysfunction, for example, infectious diseases, neurodegenerative diseases, inflammatory diseases, and lysosomal diseases with a mitochondrial fission inhibitor such as cilnidipine or a cilnidipine derivative. Exemplary non-cancer indications are described in Section 5.4.2. The methods of treating non-cancer diseases can in some embodiments comprise administering an amount of an agent or combination of agents effective to improve one or more symptoms of the disease. The methods of treating non-cancer diseases can in some embodiments comprise administering an amount of an agent or combination of agents effective to improve one or more biomarkers of the disease.

In further aspects, the disclosure provides agents and combinations of agents for use in the methods described herein, and pharmaceutical compositions containing such agents. Exemplary agents, combinations of agents and pharmaceutical compositions are described in Sections 5.1 to 5.3.

In some embodiments of the methods of the disclosure, the subject has phagocytic deficiency. Phagocytic deficiency can be measured, for example, using an in vitro assay to measure phagocytic activity. Example 1 describes an in vitro assay that can be adapted for measuring the phagocytic activity of a subject (e.g., a subject's macrophages and/or other types of phagocytes can be isolated and used in the assay as described in Example 1, but performed in the absence of cilnidipine). The phagocytic activity of a subject's phagocytes can be compared to a control phagocytic activity value, for example, obtained using phagocytes from a population of healthy subjects.

Subjects treated by the methods described herein are preferably mammals, most preferably humans.

### 5.1. Phagocytosis Activating Agents

Various phagocytosis activating agents can be used in the methods of the disclosure. For example, the agent can be a mitochondrial fission inhibitor (e.g., a Drp1 inhibitor), a PGC1α activator, an inhibitor of the PI3K-AKT-mTOR pathway, or a single component or combination of components from the culture medium of a lactic acid releasing bacteria. Combinations of such agents can also be used (e.g., a combination of two or more mitochondrial fission inhibitors, a combination of a mitochondrial fission inhibitor and a PGC1α activator, a combination of a mitochondrial fission inhibitor and an inhibitor of the PI3K-AKT-mTOR pathway, etc. can be used). It should be understood that classification of a phagocytosis activating agent as a particular type of agent is for convenience and that agents can have more than one mechanism of action. Thus, it should be understood that the methods of the disclosure comprising administration of a particular agent to a subject are not limited to a particular mechanism of action.

### 5.1.1. Mitochondrial Fission Inhibitors

Mitochondria are organelles found in almost all eukaryotic cells, and principally take part in ATP generation as a result of oxidative phosphorylation that occurs in the electron transfer system. Mitochondria undergo repeated fusion and division, and abnormality thereof is known to be involved in cancer. Mitochondrial division is induced by activation of Drp1, which is a GTP-binding protein. Cilnidipine, which functions to block the L-type calcium channel and the N-type calcium channel and has been used as a drug for the treatment of hypertension, has also been found to inhibit formation of Drp1-filamin complexes, thereby inhibiting mitochondrial fission. Nishimura et al., 2018, Sci. Signal. 11 (556): eaat5185. Example 1 of the present disclosure shows that cilnidipine also enhances the phagocytic activity of macrophages. Accordingly, in some embodiments, the phagocytosis activating agent or combination of phagocytosis activating agents comprises a Drp-1 inhibitor such as cilnidipine or a derivative thereof.

Cilnidipine has the following structure:

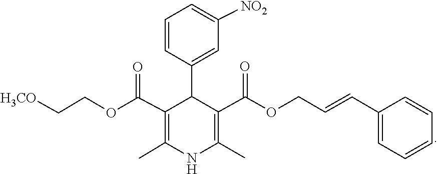 [SMILES: COCCOC(=O)C1=C(C)NC(C)=C(C(=O)OC/C=C/c2ccccc2)C1c1cccc([N+](=O)[O-])c1]

Exemplary cilnidipine derivatives are described in WO 2020/241638, the contents of which are incorporated herein by reference in their entireties.

In some embodiments, the phagocytosis activating agent is a compound of Formula (I):

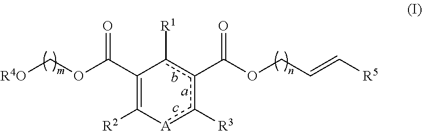

or a pharmacologically acceptable salt thereof or a solvate of them, wherein:
R¹ is phenyl substituted with one to three substituents each of which is independently NO₂, NH₂, OH, C₁-C₆ alkyl, C₁-C₆ haloalkyl, or C₁-C₆ alkoxyalkyl, provided that at least one substituent is NO₂ or NH₂;
   R² is H, C₁-C₆ alkyl, C₁-C₆ haloalkyl, or C₁-C₆ alkoxyalkyl;
   R³ is H, C₁-C₆ alkyl, C₁-C₆ haloalkyl, or C₁-C₆ alkoxyalkyl;
   R⁴ is C₁-C₆ alkyl or C₁-C₆ haloalkyl;
   R⁵ is phenyl or pyridinyl, wherein the phenyl or pyridinyl is unsubstituted or substituted with one to three substituents each of which is independently NO₂, NH₂, OH, C₁-C₆ alkyl, C₁-C₆ haloalkyl, or C₁-C₆ alkoxyalkyl;
   either (i) bond a is present and bonds b and c are absent or (ii) bonds b and c are present and bond a is absent;
   A is NH when bond a is present and N when bonds b and c are present;
   m is an integer from 1 to 4; and
   n is an integer from 1 to 3.

In some embodiments, the compound of Formula (I) is other than cilnidipine. Such compounds of Formula (I) are examples of cilnidipine derivatives.

Further features of compounds of Formula (I) are described in specific embodiments 9 to 50, infra.

In some embodiments, the compound of Formula (I) is

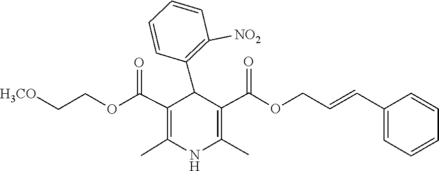 [SMILES: COCCOC(=O)C1=C(C)NC(C)=C(C(=O)OC/C=C/c2ccccc2)C1c1ccccc1[N+](=O)[O-]]

(referred to as “NS4-019” in WO 2020/241638), or a pharmacologically acceptable salt thereof or a solvate of them.

In other embodiments, the compound of Formula (I) is

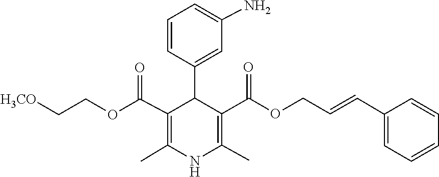 [SMILES: COCCOC(=O)C1=C(C)NC(C)=C(C(=O)OC/C=C/c2ccccc2)C1c1cccc(N)c1]

(referred to as “NS4-238” in WO 2020/241638), or a pharmacologically acceptable salt thereof or a solvate of them.

In other embodiments, the compound of Formula (I) is

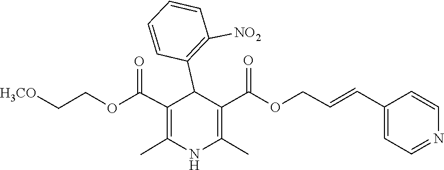 [SMILES: COCCOC(=O)C1=C(C)NC(C)=C(C(=O)OC/C=C/c2ccncc2)C1c1ccccc1[N+](=O)[O-]]

(referred to as “NS4-043” in WO 2020/241638), or a pharmacologically acceptable salt thereof or a solvate of them.

In other embodiments, the compound of Formula (I) is

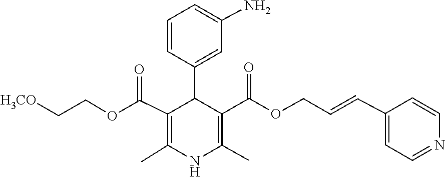 [SMILES: COCCOC(=O)C1=C(C)NC(C)=C(C(=O)OC/C=C/c2ccncc2)C1c1cccc(N)c1]

(referred to as “NS4-700” in WO 2020/241638), or a pharmacologically acceptable salt thereof or a solvate of them.

In other embodiments, the compound of Formula (I) is

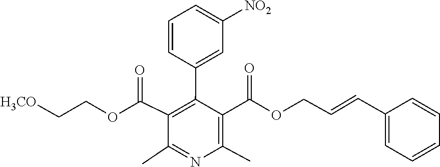 [SMILES: COCCOC(=O)c1c(C)nc(C)c(C(=O)OC/C=C/c2ccccc2)c1-c1cccc([N+](=O)[O-])c1]

(referred to as “NS4-021” in WO 2020/241638), or a pharmacologically acceptable salt thereof or a solvate of them.

In other embodiments, the compound of Formula (I) is

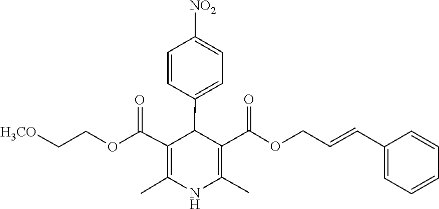 [SMILES: COCCOC(=O)C1=C(C)NC(C)=C(C(=O)OC/C=C/c2ccccc2)C1c1ccc([N+](=O)[O-])cc1]

(referred to as “JYK-002” in WO 2020/241638), or a pharmacologically acceptable salt thereof or a solvate of them.

In other embodiments, the compound of Formula (I) is

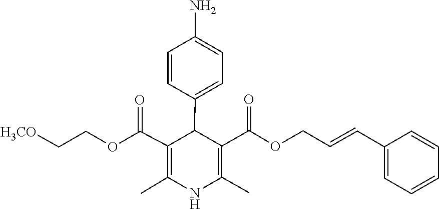 [SMILES: COCCOC(=O)C1=C(C)NC(C)=C(C(=O)OC/C=C/c2ccccc2)C1c1ccc(N)cc1]

(referred to as “JYK-003” in WO 2020/241638), or a pharmacologically acceptable salt thereof or a solvate of them.

In other embodiments, the compound of Formula (I) is

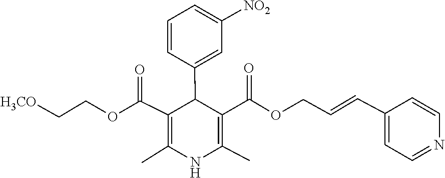 [SMILES: COCCOC(=O)C1=C(C)NC(C)=C(C(=O)OC/C=C/c2ccncc2)C1c1cccc([N+](=O)[O-])c1]

(referred to as “JYK-001” in WO 2020/241638), or a pharmacologically acceptable salt thereof or a solvate of them.

In other embodiments, the compound of Formula (I) is

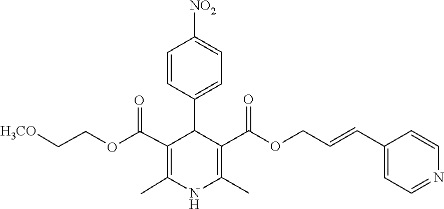 [SMILES: COCCOC(=O)C1=C(C)NC(C)=C(C(=O)OC/C=C/c2ccncc2)C1c1ccc([N+](=O)[O-])cc1]

(referred to as “JYK-004” in WO 2020/241638), or a pharmacologically acceptable salt thereof or a solvate of them.

Additional mitochondrial fission inhibitors include P110 (YGRKKRRQRRRGGDLLPRGSNH₂; see, Qi et al., 2013, J Cell Sci, 126 (Pt 3):789-802; U.S. Pat. No. 10,245,297; WO 2018/195491), metformin, mdivi-1, or berberine. Additional mitochondrial fission inhibitors, including Drp1 inhibitors, are described in WO 2012/158624, WO 2016/130143, WO 2018/052891 and WO 2019/126179, the contents of each of which are incorporated herein by reference in their entireties.

### 5.1.2. Additional Phagocytosis Activating Agents

Additional phagocytosis activating agents that can be used in the methods of the disclosure (e.g., singly or in combination with an agent described in 5.1.1 and/or and agent described in 5.1.2) include agents that activate (e.g., by virtue of being an agonist of) toll-like receptors (TLRs), Dectin-1, Mannose receptor, Scavenger receptor A, CD14, CD36, an opsonic receptor, or an apoptotic body receptor.

TLR agonists activate TLRs, including for example, TLR3, TLR4, or RIG1. Examples of TLR agonists include pathogen-associated molecular patterns (PAMPs) and mimetics thereof. These microbial molecular markers may be composed of proteins, carbohydrates, lipids, nucleic acids and/or combinations thereof, and may be located internally or externally, as known in the art. Examples include LPS, zymosan, peptidoglycans, flagellin, synthetic TLR2 agonist Pam3cys, Pam3CSK4, MALP-2, Imiquimod, CpG ODN, and the like.

TLR3 agonists include double-stranded RNA; Poly(I:C), Poly(A.U), etc., where such nucleic acids usually have a size of at least about 10 bp, at least about 20 bp, at least about 50 bp and may have a high molecular weight of from about 1 to about 20 kb, usually not more than about 50 to 100 kb. Alternative TLR3 agonists may directly bind to the protein, e.g. antibodies or small molecules that selectively bind to and activate TLR3. Other TLR3 agonists include retroviruses, e.g. a retrovirus engineered to lack the ability to integrate into the genome.

TLR3, 4, 7/8 and 9 agonists include: 852A: Synthetic imidazoquinoline mimicking viral ssRNA; VTX-2337: Small-molecule selective TLR8 agonist mimicking viral ssRNA; BCG: Bacillus of Calmette-Guerin, Mycobacterium bovis; CpG ODN: CpG oligodeoxynucleotide; Imiquimod: Synthetic imidazoquinoline mimicking viral ssRNA; LPS: Lipopolysaccharide; MPL: Monophosphoryl lipid A; Poly I:C: Polyriboinosinic-polyribocytidylic acid; PolyICLC: Poly I:C-poly-l-lysine; Resiquimod: Synthetic imidazoquinoline mimicking viral ssRNA.

Imiquimod is a synthetic imidazoquinoline that targets TLR7. A newer imidazoquinoline TLR7 agonist, 852A, administered parenterally as monotherapy has shown modest clinical efficacy with disease stabilization as a monotherapy. Resiquimod is an imidazoquinoline TLR7/8 agonist in humans.

CpG are single-strand oligodeoxynucleotides (ODNs), characterized by motifs containing cytosines and guanines. Based on their immunologic effects, CpG ODNs are divided into three different classes: CpG-A, a potent stimulator of NK cells owing to its IFN-a-producing effect on pDCs; CpG-B, a moderate IFN-a inducer, and enhancer of antigen-specific immune responses (upregulates costimulatory molecules on pDCs and B cells, induces Th 1 cytokine production and stimulates antigen presentation by pDCs); and CpG-C, which combines the stimulatory capacity of both CpG-A and CpG-B. CpG 7909 (PF-3512676, a CpG type B and TLR9 agonist) has been evaluated in several tumor types including renal cell carcinoma, glioblastoma, melanoma, cutaneous T-cell lymphoma and non-Hodgkin's lymphoma.

Polyriboinosinic-polyribocytidylic acid (Poly I:C) is a synthetic analog of viral dsRNA that stimulates endosomal (TLR3) and/or cytosolic melanoma differentiation-associated gene 5 (MDA5), leading to increased production of type I IFNs.

Lipid A molecules that target the TLR4 complex include monophosphoryl lipid A (MPL), a derivative of lipid A from Salmonella minnesota.

Dectin-1 agonists include beta-glucan, heat-killed Candida albicans, heat-killed Saccharomyces cerevisiae, whole glucan particles (WGP) and Zymosan (cell wall preparation of S. cerevisiae).

Mannose receptor activators include RP-182 (Jaynes et al., 2020, Science Translational Medicine 12 (530):eaax6337).

Scavenger receptor A activators include low endotoxin acetylated LDL (AcLDL) (Józefowski et al., 2014, Innate Immun. 20 (8):826-47).

CD14 activators include a flavivirus NS1 protein, LPS, and oxPAPC (oxidized 1-palmitoyl-2-arachidonyl-sn-glycero-3-phosphorylcholine).

CD36 activators include NKS-3 (see, WO 2019/229005) and ABT-510, a CD36 agonist peptide.

Opsonic receptor activators include aggregated C3 protein and aggregated C3b protein.

Further examples of phagocytosis activating agents include 1,3-beta glucan, mannan, lipopolysaccharide (LPS), lipoteichoic acid, lipopolysaccharide-binding protein, Plasmodium falciparum-infected erythrocytes, an IgG, an IgA. an IgE, and phosphatidylserine. In some embodiments, the phagocytosis activating agent or a combination of phagocytosis activating agents comprises 1,3-beta glucan. In some embodiments, the phagocytosis activating agent or a combination of phagocytosis activating agents comprises mannan. In some embodiments, the phagocytosis activating agent or a combination of phagocytosis activating agents comprises lipopolysaccharide (LPS). In some embodiments, the phagocytosis activating agent or a combination of phagocytosis activating agents comprises lipoteichoic acid. In some embodiments, the phagocytosis activating agent or a combination of phagocytosis activating agents comprises lipopolysaccharide-binding protein. In some embodiments, the phagocytosis activating agent or a combination of phagocytosis activating agents comprises Plasmodium falciparum-infected erythrocytes. In some embodiments, the phagocytosis activating agent or a combination of phagocytosis activating agents comprises an IgG. In some embodiments, the phagocytosis activating agent or a combination of phagocytosis activating agents comprises an IgA. In some embodiments, the phagocytosis activating agent or a combination of phagocytosis activating agents comprises an IgE. In some embodiments, the phagocytosis activating agent or a combination of phagocytosis activating agents comprises phosphatidylserine.

In further embodiments, the phagocytosis activating agent comprises an agent that activates PPARG coactivator 1 alpha (PGC1α). Exemplary PGC1α activators include metformin, ZLN005 (Zhang et al., 2013, Diabetes. 62 (4):1297-307), and Mogroside VI B (Niu et al., 2017, J Nat Prod. 80 (5):1428-1435). Additional agents that increase PGC1α activity include peroxisome proliferator-activated receptor (PPAR)-gamma agonists, AMPK activators, and sirtuin activator. Exemplary PPAR-γ agonists include thiazolidinediones (TZDs), aleglitazar, farglitazar, muraglitazar, and tesaglitazar. Exemplary TZDs include pioglitazone, rosiglitazone, rivoglitazone, and troglitazone. Exemplary AMPK activators include 5-aminoimidazole-4-carboxamide ribonucleotide (AICAR). Exemplary sirtuin activators include resveratrol, SRT1720, SRT2104, SRT2183, SRT1460, and combinations thereof.

In further embodiments, the phagocytosis activating agent or a combination of phagocytosis activating agents comprises an agent that inhibits the PI3K-AKT-mTOR pathway. The PI3K-AKT-mTOR pathway is an intracellular signaling pathway important in regulating the phagocytic capacity of macrophages (Weichhart et al., 2015, Nat Rev Immunol. 15 (10): 599-61). An agent that inhibits the PI3K-AKT-mTOR pathway can be, for example, an agent inhibits PI3K, an agent that inhibits AKT, or an agent that inhibits mTOR. Exemplary PI3K inhibitors include pictilisib, buparlisib, idelalisib, copanlisib, duvelisib, gedatolisib, and apitolisib. Exemplary AKT inhibitors include ipatasertib, MK-2206, and ARQ-092. Exemplary mTOR inhibitors include rapamycin, everolimus, 32-deoxorapamycin, 16-pent-2-ynyloxy-32-deoxorapamycin, 16-pent-2-ylyloxy-32(S)-dihydro-rapamycin, 16-pent-2-ylyloxy-32(S)-dihydr-o-40-O-(2-hydroxyethyl)-rapamycin, 40-O-(2-hydroxyethyl)-rapamycin, rapamycin 42-ester with 3-hydroxy-2-(hydroxymethyl)-2-methylpropionic acid (CCI-779), 40-[3-hydroxy-2-(hydroxymethyl)-2-methylpropanoate]-rapamycin, or a pharmaceutically acceptable salt thereof, as disclosed in U.S. Pat. No. 5,362,718, ABT578, or 40-(tetrazolyl)-rapamycin, 40-epi-(tetrazolyl)-rapamycin, e.g., as disclosed in WO 99/15530, or rapamycin analogs as disclosed in WO 98/02441 and WO 01/14387, e.g., AP23573.

In further embodiments, the phagocytosis activating agent comprises one or more bacterial strains, e.g., one or more probiotic strains and/or a component or a combination of components from a bacterial culture medium, e.g., a probiotic culture medium. Probiotic bacteria can activate phagocytosis. See, e.g., Gorska et al., 2019, Curr Microbiol. 76 (8):939-949. The bacteria can comprise, for example, a lactic acid producing bacteria. In some embodiments, the phagocytosis activating agent comprises a component of or a combination of components of a cell free extract from a lactic acid releasing bacteria culture medium. Exemplary lactic acid releasing bacteria include Lactobacillus, Leuconostoc, Pediococcus, Lactococcus, and Streptococcus, Aerococcus, Carnobacterium, Enterococcus, Oenococcus, Sporolactobacillus, Tetragenococcus, Vagococcus, and Weissella. In some embodiments, the lactic acid bacteria comprises Bifidobacterium sp. and/or Lactobacillus, e.g., L. acidophilus. Exemplary formulations of bacteria are described in WO 2016/196605 and WO 2004/087178, the contents of which are incorporated herein by reference in their entireties. A formulation comprising live bacteria can be administered by any suitable route, for example orally, by intravenous injection or by intratumoral injection.

### 5.2. Second Agents and Combinations of Second Agents

In some embodiments, a phagocytosis activating agent or combination of phagocytosis activating agents is administered in combination with one or more additional agents, for example one or more standard of care therapies for the subject's disease or one or more additional agents described herein.

Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This can be referred to as “simultaneous” or “concurrent delivery”. The term “concurrently” is not limited to the administration of therapies (e.g., a phagocytosis activating agent and an additional agent) at exactly the same time, but rather it is meant that a pharmaceutical composition comprising one agent is administered to a subject in a sequence and within a time interval such that the agent can act together with the additional agent(s) to provide an increased benefit than if they were administered otherwise. For example, each agent of a combination may be administered to a subject at the same time or sequentially in any order at different points in time; however, if not administered at the same time, they should be administered sufficiently close in time so as to provide the desired therapeutic effect.

Methods for treating cancer disclosed herein can comprise administration of a phagocytosis activating agent in combination with a CD47 inhibitor, for example an anti-CD47 antibody and/or a SIRPα inhibitor, for example, an anti-SIRPα antibody.

Phagocytosis activating agents, such as mitochondrial fission inhibitors such as cilnidipine and cilnidipine derivatives, e.g., as described in Section 5.1.1, can be used in combination with one or more additional anti-cancer therapies, e.g., a standard of care therapy for the particular cancer being treated. For example, a subject having pancreatic cancer can be treated with cilnidipine, a salt thereof, or solvate of them, in combination with gemcitabine.

### 5.2.1. CD47 Inhibitors

CD47 inhibitors include anti-CD47 antibodies, for example AO-176 (see, Puro et al., 2020, Molecular Cancer Therapeutics 19 (3):835-846), Magrolimab (also known as hu5F9-G4; Gilead), CC-90002 (Celgene; see, Zeidan et al., 2019, Blood 134 (Supplement_1): 1320), IBI-188 (Innovent Biologics), SHR-1063 (Gao et al., 2019, J Pharm Biomed Anal 175:112792, and AMMS4-G4 (Zeng, et al. 2016, Oncotarget 7: 83040-50). Further examples of anti-CD47 antibodies include clones B6H12, 5F9, 886, and C3 (for example as described in WO 2011/143624). Further anti-CD47 antibodies are described in WO 2015/191861 and WO 2016/081423. Anti-CD47 antibodies can be, for example, fully human, humanized or chimeric versions of such antibodies. Humanized antibodies (e.g., hu5F9-G4) are especially useful for in vivo applications in humans due to their low antigenicity.

Further CD47 inhibitors include TTI-621, a recombinant fusion protein composed of the human SIRPα N-terminus linked to the Fc of IgG1, ALX148, another SIRPα-Fc fusion protein (ALX Oncology) and CV1, a SIRPα variant with high affinity for CD47 that competes with endogenous SIRPα for CD47 binding (Huang et al., 2017, J Thorac Dis. 2017 February; 9 (2): E168—E174).

### 5.2.2. SIRPα Inhibitors

SIRPα inhibitors include anti-SIRPα antibodies, for example, KWAR23 (Ring et al., 2017, Proc Natl Acad Sci 114 (49):E10578-E10585), CC-95251 (Celgene), BI 765063 (also known as OSE-172; OSE Immunotherapeutics). Further examples of anti-SIRPα antibodies are described in WO 2019/023347, WO 2013/056352, and WO 2018/190719.

In some embodiments, the SIRPα inhibitor is a soluble CD47 polypeptide, e.g., as described in US 2010/0239579. In certain embodiments, a soluble CD47 polypeptide comprises the extracellular domain of CD47, including the signal peptide (SEQ ID NO:2 of WO 2016/118754), such that the extracellular portion of CD47 is typically 142 amino acids in length, and has the amino acid sequence set forth in SEQ ID NO:3 of WO 2016/118754. The soluble CD47 polypeptides described herein also include CD47 extracellular domain variants that comprise an amino acid sequence at least 65%-75%, 75%-80%, 80-85%, 85%-90%, or 95%-99% (or any percent identity not specifically enumerated between 65% to 100%), which variants retain the capability to bind to SIRPα without stimulating SIRPα signaling.

### 5.2.3. Further Agents

The phagocytosis activating agents described herein, for example, a mitochondrial fission inhibitor, can be administered as monotherapy. Alternatively, a phagocytosis activating agent can be administered to a subject with an additional agent or combination of agents for treating the subject's disease. For example, a subject suffering from a cancer can be administered one or more chemotherapeutic agents and/or one or more immunotherapeutic agents, e.g., an anti-PD1 antibody (e.g., cemiplimab, nivolumab, or pembrolizumab) and/or anti-PD-L1 antibody (e.g., avelumab, durvalumab, or atezolizumab). In some embodiments, the additional agent or combination of agents comprises a standard of care therapy for the subject's disease.

For subjects having pancreatic cancer, an additional agent or combination of agents can include, for example, gemcitabine and/or pacritaxel (paclitaxel).

For subjects having lung adenocarcinoma, an additional agent or combination of agents can include, for example, cisplatin and/or carboplatin.

For subjects having colorectal cancer, an additional agent or combination of agents can include, for example, cetuximab and/or panitumumab.

### 5.3. Pharmaceutical Compositions

The disclosure provides pharmaceutical compositions comprising one or more agents as described herein. The pharmaceutical compositions can be used in the methods of the disclosure. Pharmaceutical compositions comprising agents described herein, e.g., as described in Sections 5.2 and 5.2, including their subparts, can be formulated according to techniques known in the art. For example, agents described herein can be combined with one or more carriers, excipients, stabilizers, or a combination thereof in the form of, e.g., lyophilized powders, slurries, aqueous solutions, lotions, or suspensions (see, e.g., Hardman et al., 2001, Goodman and Gilman's The Pharmacological Basis of Therapeutics, McGraw-Hill, New York, N.Y.; Gennaro, 2000, Remington: The Science and Practice of Pharmacy, Lippincott, Williams, and Wilkins, New York, N.Y.; Avis, et al. (eds.),1993, Pharmaceutical Dosage Forms: General Medications, Marcel Dekker, NY; Lieberman, et al. (eds.), 1990, Pharmaceutical Dosage Forms: Tablets, Marcel Dekker, NY; Lieberman, et al. (eds.), 1990, Pharmaceutical Dosage Forms: Disperse Systems, Marcel Dekker, NY; Weiner and Kotkoskie, 2000, Excipient Toxicity and Safety, Marcel Dekker, Inc., New York, N.Y.).

In one aspect, the disclosure provides a pharmaceutical composition comprising a phagocytosis activating agent as described herein and/or an additional agent or combination of additional agents and a pharmacologically acceptable diluent, carrier, or excipient.

The content amount of phagocytosis activating agent and/or an additional agent or combination of additional agents in the pharmaceutical composition can be appropriately selected and is generally in the range of 0.01 to 100% by weight (e.g., 1% to 99%, 1% to 90%, 5% to 80%, 10% to 75%, or 15% to 50% by weight of the pharmaceutical composition, or any weight percent range bound by any two of the foregoing values).

A phagocytosis activating agent (e.g., a mitochondrial fission inhibitor) and/or an additional agent or combination of agents can be formulated in a pharmaceutical composition comprising one or more agents designed to target delivery of the phagocytosis activating agent and/or additional agent or combination of additional agents to the tumor microenvironment and/or phagocytes. For example, an agent or combination of agents can be formulated with a bile acid and/or bile acid derivative, for example ursodeoxycholic acid. Such formulations can be used, for example, to enhance delivery of an agent which is an orally available hydrophobic small molecule. See, Lozano et al., 2015, J Control Release 216:93-102; Mooranian et al., 2020, Sci Rep 10:106; Pavlovic et al., 2018, Front. Pharmacol. 9:1283; U.S. Pat. No. 10,765,751, the contents of each of which are incorporated herein by reference in their entireties.

In additional aspects, the disclosure provides pharmaceutical compositions comprising phagocytes (e.g., monocytes, macrophages, neutrophils, dendritic cells, mast cells, or any combination thereof) having enhanced phagocytic activity. Enhanced phagocytic activity can be measured, for example, as described in Example 1. The pharmaceutical compositions comprising phagocytes can contain, for example, a population of phagocytes and one or more additional components, for example, a buffer or cell culture medium.

Phagocytes having enhanced phagocytic activity can be obtained by treating phagocytes ex-vivo with a mitochondrial fission inhibitor, for example cilnidipine and/or another mitochondrial fission inhibitor described in Section 5.1.1. Such pharmaceutical compositions can be used in methods of treating a subject having a phagocytosis deficiency-related disease or condition, for example a cancer or other disease or condition disclosed in Section 5.4. The phagocytes can be autologous to the subject to be treated or, alternatively, the phagocytes can be allogeneic to the subject to be treated.

A composition of the present disclosure may also be administered via one or more routes of administration using one or more of a variety of methods known in the art. As will be appreciated by the skilled artisan, the route and/or mode of administration will vary depending upon the desired results. Selected routes of administration include intravenous, intramuscular, intradermal, intraperitoneal, subcutaneous, spinal or other general routes of administration, for example by injection or infusion. General administration may represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal, intratumoral, peritumoral, intralymphatic, injection and infusion. Alternatively, a composition of the disclosure can be administered via a non-general route, such as a topical, epidermal or mucosal route of administration, for example, intranasally, orally, vaginally, rectally, sublingually or topically.

In another aspect, the disclosure provides uses of the agents disclosed herein, for example, cilnidipine or a pharmacologically acceptable salt thereof or a solvent of them, in the manufacture of a medicament for treating a disease or condition disclosed herein, e.g., pancreatic cancer or DMD), optionally wherein the medicament is formulated for administration as monotherapy or as part of a combination therapy regimen, e.g., with a second agent as described herein.

### 5.4. Cancers and Phagocytosis Deficiency-related Diseases and Conditions

### 5.4.1. Cancers

Cancers that can be treated according to the methods of the disclosure include hematological cancers and solid tumors. Exemplary cancers include pancreatic cancer (e.g., pancreatic ductal adenocarcinoma), lung cancer, small cell lung cancer or non-small cell lung cancer, e.g., lung adenocarcinoma, colorectal cancer, melanoma (e.g., having a BRAF mutation), leukemia, e.g., acute myeloid leukemia or acute lymphocytic leukemia, lymphoma, e.g., non-Hodgkin lymphoma, diffuse large B-Cell lymphoma (DLBCL),myeloma, e.g., multiple myeloma, leiomyosarcoma, breast cancer, liver cancer, osteosarcoma, and head and neck cancer. In some embodiments, the subject has acute myeloid leukemia, lymphoma, non-Hodgkin lymphoma, or DLBCL.

In some embodiments, the cancer has a KRAS mutation. KRAS is a protein involved in cell signaling pathways that control cell growth, cell maturation, and cell death. Mutated forms of the KRAS gene have been found in some types of cancer, including non-small cell lung cancer (NSCLC), colorectal cancer, lung cancer, and pancreatic cancer. These mutations can drive cancer cells to grow and spread in the body.

Without being bound by theory, it is believed that Drp1 de-activation by a mitochondrial fission inhibitor, such as a Drp1 inhibitor, for example, cilnidipine, can inhibit the proliferative signal caused by KRAS mutations in tumor cells. It is further believed that Drp1 inhibition can cause a metabolic shift from aerobic glycolysis to fatty acid oxidation, reducing the oxidative stress in the tumor microenvironment, facilitating CD8+ T-cell infiltration into the tumor, and/or increasing T-cell durability in the tumor microenvironment. Again without being bound by theory, it is believed that the metabolic shift can recover phagocytic capability of macrophages and natural killer cells in the tumor microenvironment, facilitating tumor depletion and/or priming/activation of T-cells.

In some aspects, the disclosure provides cilnidipine or a pharmacologically acceptable salt thereof or a solvate of them for use in a method of treating pancreatic cancer, e.g., pancreatic ductal adenocarcinoma. In some embodiments, cilnidipine or a pharmacologically acceptable salt thereof or a solvate of them is administered orally. In some embodiments, the cilnidipine or a pharmacologically acceptable salt thereof or a solvate of them is administered as monotherapy. In other embodiments, the cilnidipine or a pharmacologically acceptable salt thereof or a solvate of them is administered in combination with one or more additional agents, for example gemcitabine (e.g., administered intravenously). In some embodiments, the amount of cilnidipine or a pharmacologically acceptable salt thereof or a solvate of them administered as monotherapy is an amount effective to slow tumor growth in the subject. In some embodiments, when the cilnidipine or a pharmacologically acceptable salt thereof or a solvate of them is administered in combination with gemcitabine, the amounts of cilnidipine and gemcitabine together are effective to slow tumor growth in the subject. In some embodiments, the amounts of cilnidipine and gemcitabine together are more effective to slow tumor growth in the subject than when administered alone.

### 5.4.2. Non-cancer Phagocytosis Deficiency-related Diseases and Conditions

Phagocytosis deficiency is associated with a number of diseases and conditions, for example, infectious diseases, neurodegenerative diseases, inflammation, inflammatory diseases, and lysosomal diseases (see, e.g., Engelich et al., 2001, Clinical Infectious Diseases 33 (12):2040-2048; Andrews and Sullivan, 2003, Clin Microbiol Rev. 16 (4):597-621; Galloway et al., 2019, Front Immunol. 10:790; Kourtzelis et al., 2020, Front Immunol. 2020; 11: 553; Maderna and Godson, 2003, Biochimica et Biophysica Acta (BBA)—Molecular Basis of Disease, 1639(3):141-151; Abdolmakeki et al., 2018, Front Immunol. 9:1645; Marques and Saftig, 2019, Journal of Cell Science 132:jcs221739). Phagocytosis also has a role in musculoskeletal degenerative diseases such as Duchenne muscular dystrophy (DMD) (see, e.g., Rosenberg et al., 2015, Sci Transl Med. 7 (299):299rv4.

Infectious diseases include diseases caused by bacteria (e.g., Streptococcal infections), viruses (e.g., influenza, hepatitis B, hepatitis C, HIV), fungi (e.g., yeast infection) or parasites (e.g., malaria). Neurodegenerative diseases include Alzheimer's disease (AD), progressive supranuclear palsy (PSP) and other dementias, Parkinson's disease (PD), Nasu-Hakola disease, prion disease, amyotrophic lateral sclerosis (ALS), Friedreich's ataxia, Huntington's disease, Lewy body disease, adrenoleukodystrophy (ALD) and spinal muscular atrophy. Exemplary inflammatory diseases include autoimmune diseases such as systemic lupus erythematosus (SLE), rheumatoid arthritis (RA), and autoimmune lymphoproliferative syndrome (ALPS). Lysosomal storage diseases include Gaucher disease, Fabry disease, Niemann-Pick disease, Hunter syndrome, Glycogen storage disease II (Pompe disease), and Tay-Sachs disease. Musculoskeletal degenerative diseases include muscular dystrophies such as DMD. Accordingly, in some embodiments, the subjects treated according to the methods of the disclosure have a non-cancer disease described herein.

In some aspects, the disclosure provides cilnidipine or a pharmacologically acceptable salt thereof or a solvate of them for use in a method of a subject with DMD. In some embodiments, cilnidipine or a pharmacologically acceptable salt thereof or a solvate of them is administered orally. In some embodiments, the cilnidipine or a pharmacologically acceptable salt thereof or a solvate of them is administered as monotherapy. In some embodiments, the amount of cilnidipine or a pharmacologically acceptable salt thereof or a solvate of them is administered is an amount effective to decrease plasma creatinine kinase (CK) and/or lactose dehydrogenase (LDH) levels.

### 6. EXAMPLES

### 6.1. Example 1: In Vitro Study to Evaluate the Effect of Cilnidipine on Phagocytosis by Macrophages

The effect of cilnidipine on phagocytosis by macrophages was evaluated in vitro using the J774.1 mouse macrophage cell line. Cells were seeded at 2×10⁵ cells/well/200 μL of a 48 well plate and cultured for 24 hours in DMEM medium containing 10% FBS and antibiotics at 37° C. under 5% CO₂. Cells were washed once and then incubated with cilnidipine at concentrations 0.3 μM, 1 μM, or 3 μM, or DMSO (vehicle) for 20 hours.

The level of phagocytosis was evaluated by adding 10 μL of DMEM medium containing 4×10⁶ PE-labeled polystyrene beads (Fluoresbrite® Polychromatic Red Microspheres 2.0 μm) to cells and culturing them for 2.5 hours. Cells were then washed 3 times with PBS and collected. The level of phagocytic activity was assessed by flow cytometry, measuring the amount of fluorescent signal derived from the PE-labelled beads that were taken up by the cells, with cells treated with 0.1% DMSO used as negative control. The proportions of cells engulfing 1 or more, 2 or more, 3 or more, and 4 or more particles were measured.

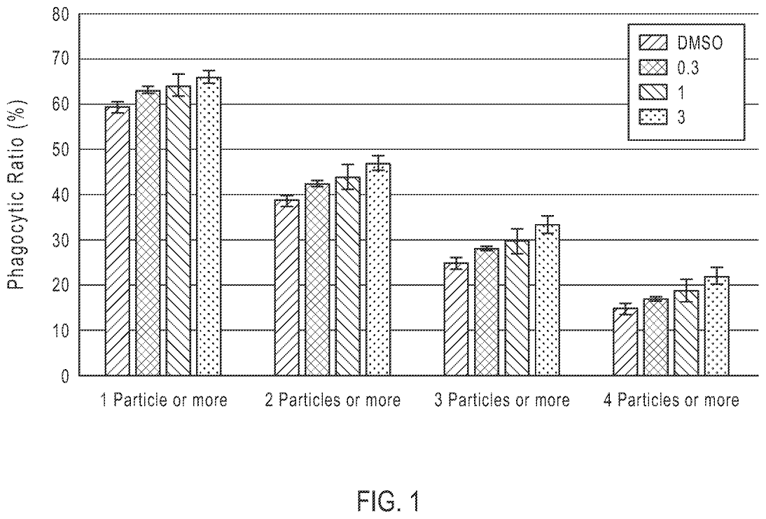

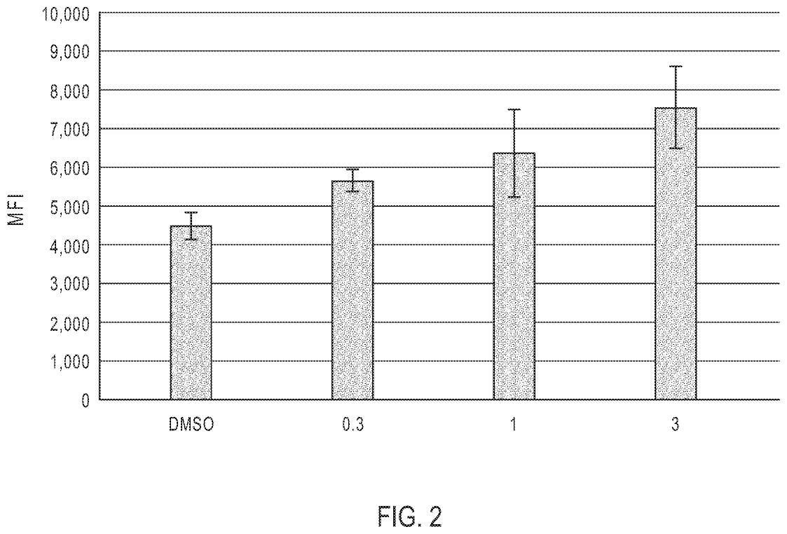

As shown in FIG. 1, cilnidipine enhanced phagocytic activity of macrophages in a dose dependent manner. Mean fluorescence intensity for each cilnidipine dose is shown in FIG. 2.

### 6.2. Example 2: Evaluation of Anti-proliferative Effects of Cilnidipine on Cancer Cell Lines In Vitro

The effect of cilnidipine on cell proliferation of eight cancer cell lines is evaluated. Five pancreatic cancer cell lines are included, four containing KRAS mutations (AsPC-1 (G12D), Capan-1 (G12V), KP4 (M188V, G12D) and MIA PaCa-2 (G12C)), and one pancreatic cancer cell line with wild type KRAS, BxPC-3, in addition to three melanoma cell lines, two of which contain B-Raf mutations: A375 (V600E) and SK-MEL-28 (V600E), and one melanoma cell line with wild type B-Raf, MeWo.

Treatment of pancreatic and melanoma cancer cell lines with cilnidipine has an anti-proliferative effect on the cell lines.

### 6.3. Example 3: In Vivo Evaluation of Anti-tumor Effects of Cilnidipine

The in vivo anti-tumor effect of cilnidipine is assessed in MC38 tumor-bearing C57BL/6 mice. Five or seven week old C57BL/6 mice are maintained under specific pathogen-free (SPF) conditions. Mice are injected subcutaneously with 5×10⁵ MC38 cells, a murine colon adenocarcinoma cell line, and treated i.t. with cilnidipine on days 10 and 14 after tumor inoculation. Tumor volume is measured at the various time points.

Treatment of mice with cilnidipine reduces tumor growth as compared to control mice.

### 6.4. Example 4: In Vivo Evaluation of Cilnidipine in a DMD Model

The effect of cilnidipine and the cilnidipine derivative NS4-043 in the mdx mouse model of Duchenne muscular dystrophy (DMD) model was evaluated. The mdx mouse has a point mutation in its DMD gene, changing the amino acid coding for a glutamine to a stop codon, which causes the muscle cells to produce a small, nonfunctional dystrophin protein. The study included cilnidipine, NS4-043, idebenone (positive control) and vehicle treatment groups as shown in Table 1.

TABLE 1

| | | | Number of | |
|---|---|---|---|---|
| Group | Treatment | Mouse Strain | animals | Dose |
| 1 | Vehicle | C57BL/10SnSlc | 8 | — |
| | | (male) | | |
| 2 | Vehicle | C57BL/10-mdx | 8 | — |
| | | (male) | | |
| 3 | Idebenone | C57BL/10-mdx | 6 | 200 mg/kg |
| | | (male) | | |
| 4 | Cilnidipine | C57BL/10-mdx | 10 | 10 mg/kg |
| | | (male) | | |
| 5 | NS4-043 | C57BL/10-mdx | 9 | 10 mg/kg |
| | | (male) | | |

Animals were dosed once daily with vehicle, idebenone or cilnidipine on study days: 0 to 20.

Mice were evaluated by a rotarod test on study days 19 and 20. Body weight was measured throughout the study period. Creatinine kinase (CK) and lactose dehydrogenase (LDH), two biomarkers of DMD, were measured on day 21.

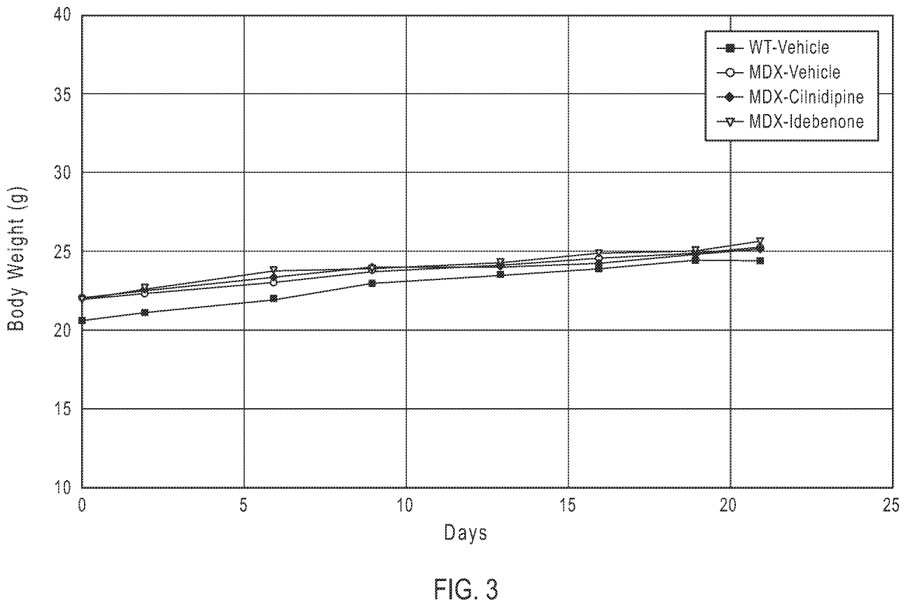

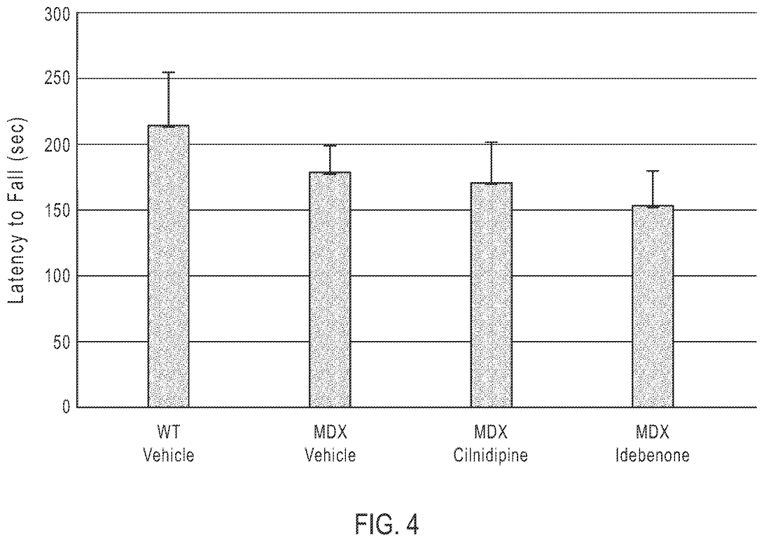

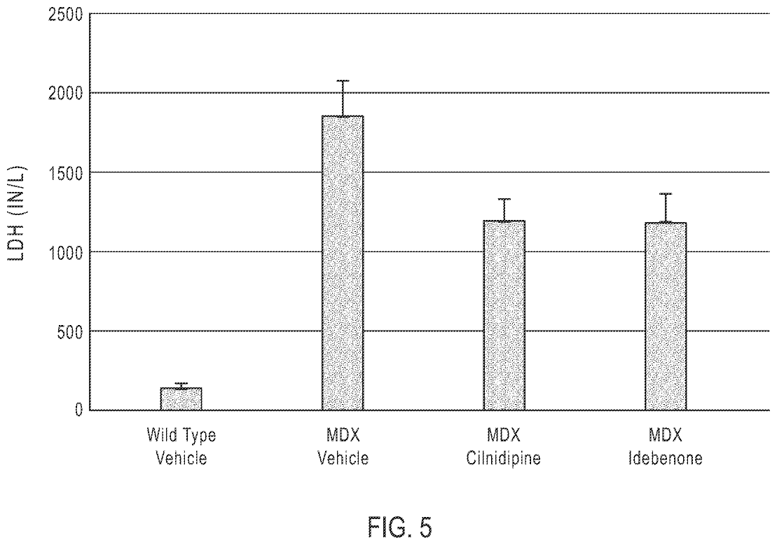

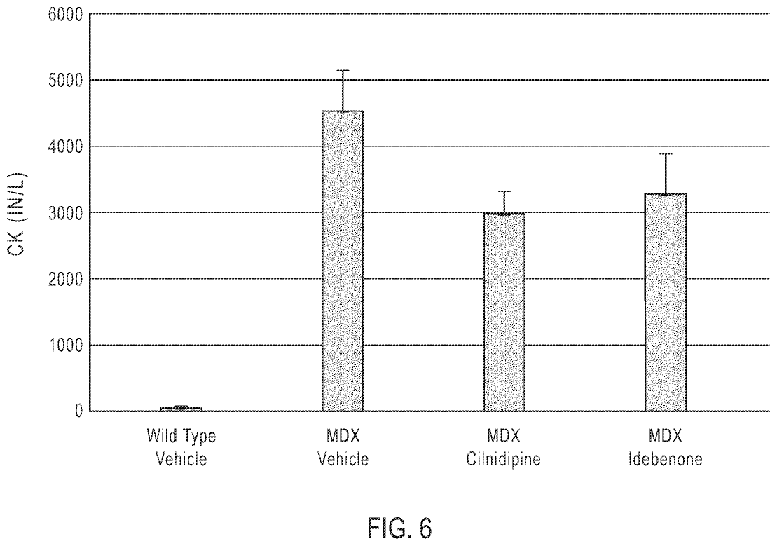

No significant effect on body weight was observed for any treatment group (FIG. 3; NS4-043 data not shown). Latency (time to fall) in the rotarod test was decreased in the mdx mice (all groups) compared to wild-type mice, but the decrease did not reach statistical significance for any group (FIG. 4; NS4-043 data not shown). LDH was significantly increased in the plasma of mdx mice compared to wild-type. Both cilnidipine and idebenone significantly decreased plasma LDH (FIG. 5). NS4-043 did not significantly decrease plasma LDH in this study (data not shown), possibly due to insufficient exposure (dosing) to drug. CK was significantly increased in the plasma of mdx mice compared to wild-type. Surprisingly, cilnidipine, but not idebenone, significantly suppressed plasma CK (FIG. 6). Plasma CK in NS4-043 treated animals was similar to plasma CK in idebenone treated animals (data not shown). Thus, this study supports the use of cilnidipine and cilnidipine derivatives such as NS4-043 as a treatment for DMD.

### 6.5. Example 5: In Vivo Evaluation of Cilnidipine and Cilnidipine in Combination with Gemcitabine in a Pancreatic Cancer Model

A study of the in vivo therapeutic efficacy of cilnidipine as a single agent or in combination with gemcitabine in the treatment of the subcutaneous Capan-1 pancreatic cancer xenograft model in female BALB/c nude mice was performed.

### 6.5.1. Materials and Methods

### 6.5.1.1. Animals

Female BALB/c nude mice 7-9 weeks of age were used in the study.

### 6.5.1.2. Study Design

The study contained six treatment groups as shown in Table 2.

TABLE 2

| | | | Dose | Dosing | | Dosing |
|---|---|---|---|---|---|---|
| | | | Level | Volume | Route of | Frequency& |
| Group | N | Treatment | (mg/kg) | (ml/kg) | administration | Duration |
| 1 | 8 | Vehicle: 5% | — | 10 | p.o. | QD × 21 |
| | | DMSO + Corn Oil | | | | days |
| | | Vehicle: saline | — | 10 | i.p. | Q3D × 4 |
| 2 | 8 | Gemcitabine | 30 | 10 | i.p. | Q3D × 4 |
| 3 | 8 | Gemcitabine | 100 | 10 | i.p. | Q3D × 4 |
| 4 | 8 | Cilnidipine | 10 | 10 | p.o. | QD × 21 |
| | | | | | | days |
| 5 | 8 | Gemcitabine | 30 | 10 | i.p. | Q3D × 4 |
| | | Cilnidipine | 10 | 10 | p.o. | QD × 21 |
| | | | | | | days |
| 6 | 8 | Gemcitabine | 100 | 10 | i.p. | Q3D × 4 |
| | | Cilnidipine | 10 | 10 | p.o. | QD × 21 |
| | | | | | | days |

N: number of animals per group
p.o.: oral
i.p.: intraperitoneal
QD: once a day
Q3D: once every three days (one day dosing and two days off)

### 6.5.1.3. Study Methods

The study contained six treatment groups as shown in Table 2.

### 6.5.1.3.1. Cell Culture

Capan-1 human pancreatic ductal adenocarcinoma cells were cultured in IMEM medium with 20% FBS at 37° C. and 5% CO₂ in air atmosphere.

### 6.5.1.3.2. Tumor Inoculation

Each mouse was inoculated subcutaneously in the right upper flank region with tumor cells (5×10⁶ cells) in 0.1 ml of PBS with Matrigel (1:1) for tumor development.

### 6.5.1.3.3. Randomization

Randomization was started when the mean tumor size reached approximately 254.27 mm³. A total of 48 mice were enrolled in the study and randomly allocated to the six study groups, with eight mice per group. Randomization was performed based on “Matched distribution” method (Study Director™ software, version 3.1.399.19). The date of randomization was denoted as day 0.

### 6.5.1.3.4. Observation and Data Collection

After tumor inoculation, the animals were checked daily for morbidity and mortality. During routine monitoring, the animals were checked for any effects of tumor growth and treatments on behavior such as mobility, food and water consumption, body weight gain/loss (body weights were measured twice per week after randomization), eye/hair matting and any other abnormalities. Mortality and observed clinical signs were recorded for individual animals in detail.

Tumor volumes were measured twice per week after randomization in two dimensions using a caliper, and the volume was expressed in mm³ using the formula: V=(L×W×W)/2, where V is tumor volume, L is tumor length (the longest tumor dimension) and W is tumor width (the longest tumor dimension perpendicular to L). Dosing as well as tumor and body weight measurements were conducted in a Laminar Flow Cabinet. Body weights and tumor volumes were measured by using Study Director™ software (version 3.1.399.19).

### 6.5.1.3.5. Drug Formulation and Test Article Administration

The vehicle used for cilnidipine was 5% DMSO and corn oil. The vehicle used for gemcitabine was saline. Dosing volume was adjusted for body weight (dosing volume=5/10 μL/g). Treatments were initiated on day 0 following randomization.

### 6.5.1.3.6. Dosing Holiday and Supplemental Gel Administration

The study was planned so that (1) mice showing a body weight loss (BWL)>20% after one measurement would be sacrificed, (2) mice showing BWL>15% after one measurement would be given a dosing holiday in a separate cage alone with daily monitoring for 72 hours, while other mice in the same group would receive dosing as scheduled, with treatment of the isolated mouse resuming when the BWL had recovered to BWL<10%, and (3) mice showing a BWL>10% would receive supplemental gel. BWL was calculated based on the BW on the first day of treatment.

### 6.5.1.4. Study Termination

### 6.5.1.4.1. Study Endpoints

The study endpoint was tumor growth inhibition (TGI). TGI% is an indication of antitumor activity. TGI is expressed as: TGI (%)=100×(1−T/C). T and C are the mean tumor volume of the treated and control groups, respectively, on a given day.

### 6.5.1.4.2. Treatment Termination

The treatment was performed for 21 days. There was a 2-week observation period without dosing after the treatment.

### 6.5.1.4.3. Humane Endpoints

Humane endpoints were based on body weight loss, tumor size, tumor appearance monitoring, and general animal welfare surveillance. The body weight of all animals was monitored throughout the study and animals would be euthanized if they lost over 20% of their body weight relative to the weight at the first day of treatment. Individual mice would be sacrificed when tumor volume exceeded 3000 mm³. To deter cannibalization, any animal exhibiting an ulcerated or necrotic tumor would be separated immediately and singly housed and monitored daily before the animal was euthanized or until tumor regression was complete. Mice with tumor ulceration of approximately 25% or greater on the surface of the tumor would be euthanized.

### 6.5.1.5. Statistical Analysis

To compare tumor volumes of different groups at a pre-specified day, Bartlett's test was used to check the assumption of homogeneity of variance across all groups. When the p-value of Bartlett's test was >=0.05, one-way ANOVA was performed to test overall equality of means across all groups. If the p-value of the one-way ANOVA was <0.05, Dunnett's tests were performed for comparing each treatment group with the vehicle group. When the p-value of Bartlett's test was <0.05, Kruskal-Wallis test was performed to test overall equality of medians among all groups. If the p-value the Kruskal-Wallis test was <0.05, post hoc testing by running Conover's non-parametric test was performed for all pairwise comparisons or for comparing each treatment group with the vehicle group, both with single-step p-value adjustment.

All statistical analyses were performed in R-a language and environment for statistical computing and graphics (version 3.3.1). All tests were two-sided unless otherwise specified, and p-values of <0.05 were regarded as statistically significant.

### 6.5.2. Results

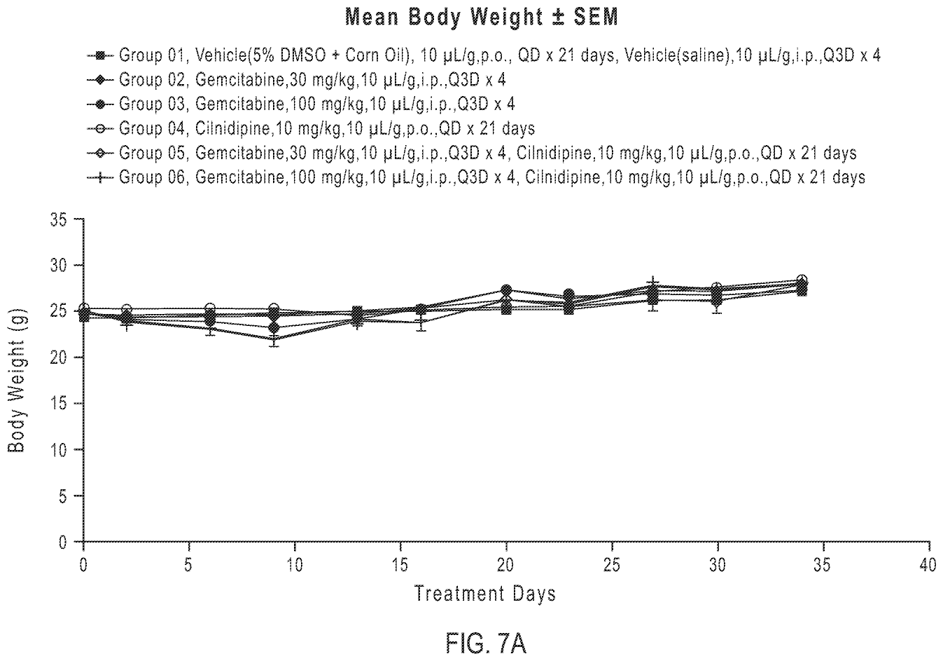

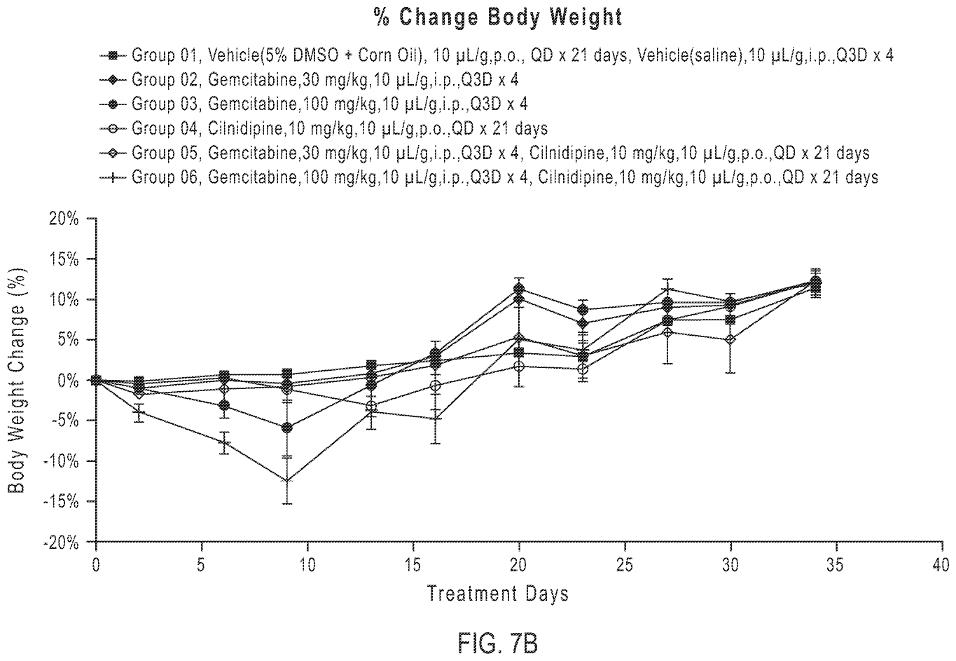

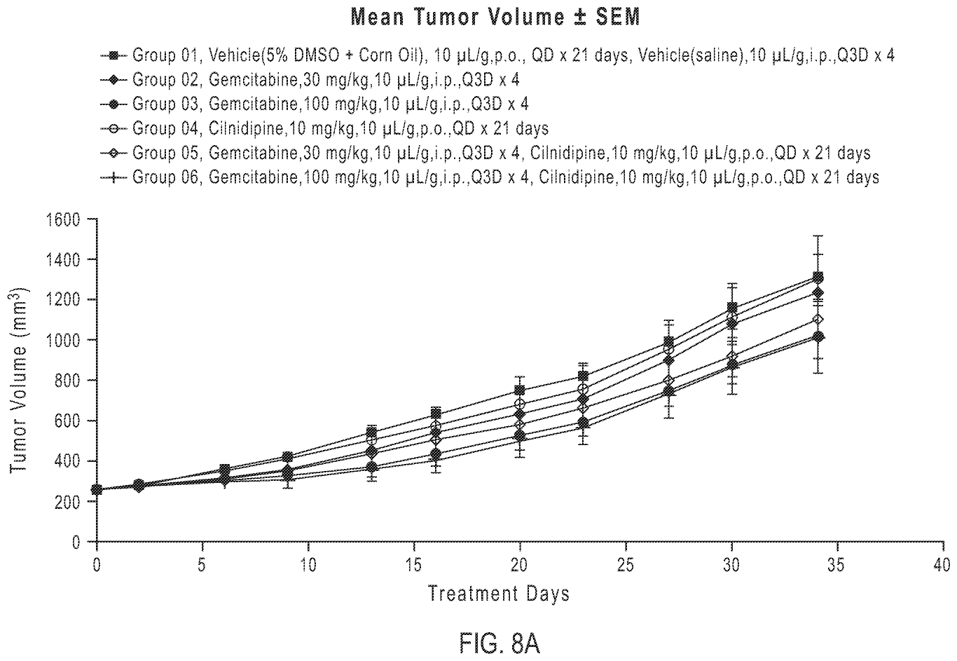

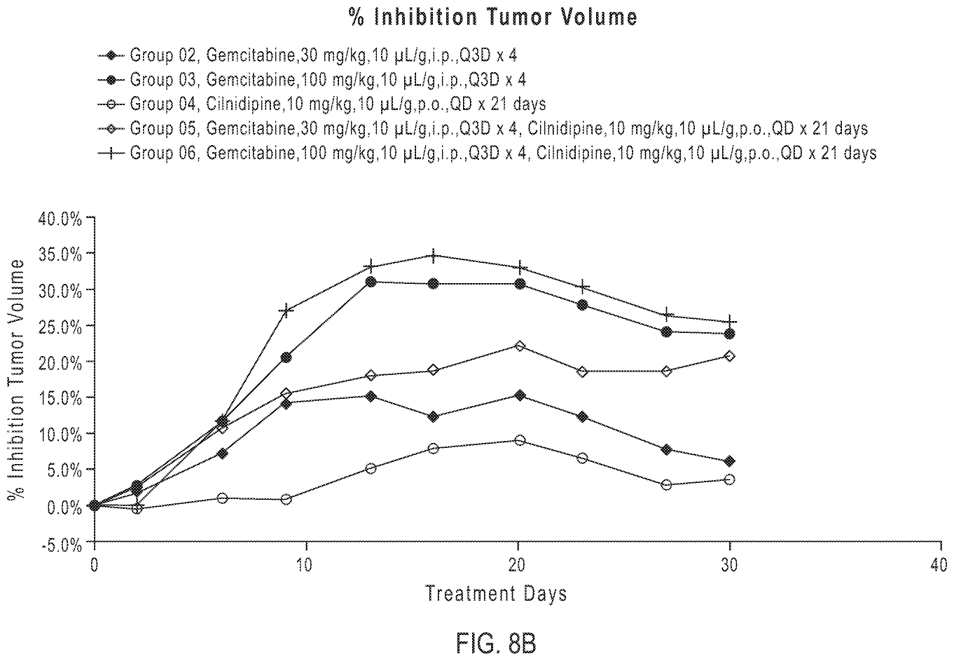

Results are shown in FIGS. 7A-7B and FIGS. 8A-8B.

No signs of cachexia were observed in vehicle group (Group 1). BWL>15% was observed in mice in Groups 3, 4, 5 and 6. Mean body weights for each group at different time points are shown in FIG. 7A. The body weight change curves of for each group at different time points are shown in FIG. 7B.

A trend of anti-tumor activity was observed for cilnidipine as a single agent (Group 4) and in combination with gemcitabine (Groups 5 and 6), where the calculated TGI at 20 days was 9.01%, 22.18% and 33.06% (p>0.05) respectively, even though the anti-tumor activity observed did not reach statistical significance (FIGS. 8A-8B and Table 3, below).

TABLE 3
Tumor Growth Inhibition (TGI) on Day 20

| Group | TGI vs G01 (%) |
|---|---|
| G01 | — |
| G02 | 15.273 |
| G03 | 30.717 |
| G04 | 9.014 |
| G05 | 22.184 |
| G06 | 33.064 |

This study supports the use of cilnidipine, alone or in combination with gemcitabine, in the treatment of cancers such as pancreatic cancer.

### 7. SPECIFIC EMBODIMENTS

The present disclosure is exemplified by the specific embodiments below.
1. A method of treating a subject having a phagocytosis deficiency-related disease or condition, which is optionally a cancer, comprising administering an agent or combination of agents to the subject comprising a phagocytosis activating agent.
2. The method of embodiment 1, wherein the agent or combination of agents comprises a mitochondrial fission inhibitor.
3. A method of treating a subject having a disease that is a cancer, comprising administering an agent or combination of agents to the subject comprising a mitochondrial fission inhibitor.
4. The method of embodiment 2 or embodiment 3, which comprises administering a combination of agents comprising two or more mitochondrial fission inhibitors.
5. The method of any one of embodiments 1 to 4, wherein the agent or combination of agents comprises a Drp1 inhibitor.
6. The method of any one of embodiments 1 to 5, wherein the agent or combination of agents comprises cilnidipine, P110, metformin, mdivi-1, berberine, a pharmacologically acceptable salt thereof or a solvate of them or a combination of any of the foregoing.
7. The method of any one of embodiments 1 to 6, wherein the agent or combination of agents comprises cilnidipine or a pharmacologically acceptable salt thereof or a solvate of them.
8. The method of any one of embodiments 1 to 7, wherein the agent or combination of agents comprise a cilnidipine derivative or a pharmacologically acceptable salt thereof or a solvate of them.
9. The method of any one of embodiments 1 to 8, wherein the agent or combination of agents comprises a compound of Formula (I):

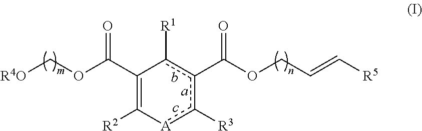

or a pharmacologically acceptable salt thereof or a solvate of them, wherein:
R¹ is phenyl substituted with one to three substituents each of which is independently NO₂, NH₂, OH, C₁-C₆ alkyl, C₁-C₆ haloalkyl, or C₁-C₆ alkoxyalkyl, provided that at least one substituent is NO₂ or NH₂;
R² is H, C₁-C₆ alkyl, C₁-C₆ haloalkyl, or C₁-C₆ alkoxyalkyl;
R³ is H, C₁-C₆ alkyl, C₁-C₆ haloalkyl, or C₁-C₆ alkoxyalkyl;
R⁴ is C₁-C₆ alkyl or C₁-C₆ haloalkyl;
R⁵ is phenyl or pyridinyl, wherein the phenyl or pyridinyl is unsubstituted or substituted with one to three substituents each of which is independently NO₂, NH₂, OH, C₁-C₆ alkyl, C₁-C₆ haloalkyl, or C₁-C₆ alkoxyalkyl;
either (i) bond a is present and bonds b and c are absent or (ii) bonds b and c are present and bond a is absent;
A is NH when bond a is present and N when bonds b and c are present;
m is an integer from 1 to 4; and
n is an integer from 1 to 3,
optionally wherein the compound of Formula (I) is not cilnidipine.
10. The method of embodiment 9, wherein the compound of Formula (I) is a compound having the structure of Formula (Ia):

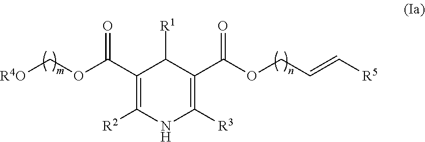

or a pharmacologically acceptable salt thereof or a solvate of them.
11. The method of embodiment 9, wherein the compound of Formula (I) is a compound having the structure of Formula (Ib):

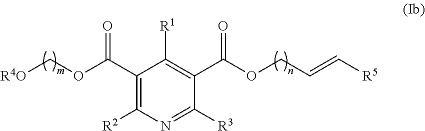

or a pharmacologically acceptable salt thereof or a solvate of them.
12. The method of any one of embodiments 9 to 11, wherein R¹ is:

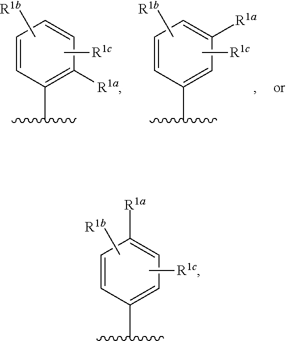

wherein R^{1a} is NO₂ or NH₂, and R^{1b} and R^{1c} are each independently H, NO₂, NH₂, OH, C₁-C₆ alkyl, C₁-C₆ haloalkyl, or C₁-C₆ alkoxyalkyl.
13. The method of embodiment 12, wherein R^{1b} and R^{1c} are each independently H, NO₂, NH₂, OH, C₁-C₃ alkyl, C₁-C₃ haloalkyl, or C₁-C₃ alkoxyalkyl.
14. The method of embodiment 12, wherein R¹ is

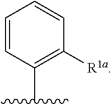

15. The method of embodiment 12, wherein R¹ is

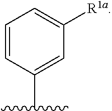

16. The method of embodiment 12, wherein R¹ is

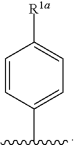

17. The method of any one of embodiments 12 to 16, wherein R^{1a} is NO₂.
18. The method of any one of embodiments 12 to 16, wherein R^{1a} is NH₂.
19. The method of any one of embodiments 9 to 18, wherein R² is H, C₁-C₃ alkyl, C₁-C₃ haloalkyl, or C₁-C₃ alkoxyalkyl.
20. The method of embodiment 19, wherein R² is C₁-C₃ alkyl.
21. The method of embodiment 20, wherein R² is CH₃.
22. The method of any one of embodiments 9 to 21, wherein R³ is H, C₁-C₃ alkyl, C₁-C₃ haloalkyl, or C₁-C₃ alkoxyalkyl.
23. The method of embodiment 22, wherein R³ is C₁-C₃ alkyl.
24. The method of embodiment 23, wherein R³ is CH₃ .
25. The method of any one of embodiments 9 to 24, wherein R⁴ is C₁-C₃ alkyl or C₁-C₃ haloalkyl.
26. The method of embodiment 25, wherein R⁴ is C₁-C₃ alkyl.
27. The method of embodiment 26, wherein R⁴ is CH₃.
28. The method of any one of embodiments 9 to 27, wherein R⁵ is phenyl which is unsubstituted or substituted with one to three substituents each of which is independently NO₂, NH₂, OH, C₁-C₆ alkyl, C₁-C₆ haloalkyl, or C₁-C₆ alkoxyalkyl.
29. The method of embodiment 28, wherein R⁵ is phenyl which is unsubstituted or substituted with one to three substituents each of which is independently NO₂, NH₂, OH, C₁-C₃ alkyl, C₁-C₃ haloalkyl, or C₁-C₃ alkoxyalkyl.
30. The method of embodiment 28, wherein R⁵ is unsubstituted phenyl.
31. The method of any one of embodiments 9 to 27, wherein R⁵ is pyridinyl which is unsubstituted or substituted with one to three substituents each of which is independently NO₂, NH₂, OH, C₁-C₆ alkyl, C₁-C₆ haloalkyl, or C₁-C₆ alkoxyalkyl.
32. The method of embodiment 31, wherein R⁵ is pyridinyl which is unsubstituted or substituted with one to three substituents each of which is independently NO₂, NH₂, OH, C₁-C₃ alkyl, C₁-C₃ haloalkyl, or C₁-C₃ alkoxyalkyl.
33. The method of embodiment 31, wherein R⁵ is unsubstituted pyridinyl.
34. The method of any one of embodiments 31 to 33, wherein the R⁵ pyridinyl is 4-pyridyl.
35. The method of any one of embodiments 31 to 33, wherein the R⁵ pyridinyl is 3-pyridyl. pyridyl.
36. The method of any one of embodiments 31 to 33, wherein the R⁵ pyridinyl is 2-pyridyl.
37. The method of any one of embodiments 9 to 36, wherein m is 2.
38. The method of any one of embodiments 9 to 36, wherein n is 1.
39. The method of embodiment 10, wherein:

### R¹ is

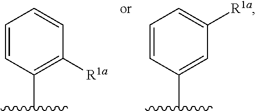

wherein R^{1a} is NO₂ or NH₂;
R², R³, and R⁴ are CH₃;
R⁵ is unsubstituted phenyl or unsubstituted pyridinyl;
m is 2; and
n is 1.
40. The method of embodiment 39, wherein R⁵ is unsubstituted 4-pyridyl.
41. The method of embodiment 9, wherein the compound of Formula (I) is cilnidipine or a pharmacologically acceptable salt thereof or a solvate of them.
42. The method of embodiment 9, wherein the compound of Formula (I) is

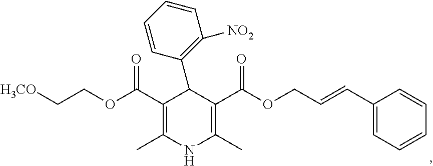 [SMILES: COCCOC(=O)C1=C(C)NC(C)=C(C(=O)OC/C=C/c2ccccc2)C1c1ccccc1[N+](=O)[O-]]

or a pharmacologically acceptable salt thereof or a solvate of them.
43. The method of embodiment 9, wherein the compound of Formula (I) is

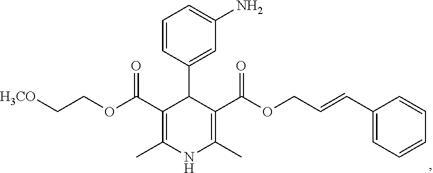 [SMILES: COCCOC(=O)C1=C(C)NC(C)=C(C(=O)OC/C=C/c2ccccc2)C1c1cccc(N)c1]

or a pharmacologically acceptable salt thereof or a solvate of them.
44. The method of embodiment 9, wherein the compound of Formula (I) is

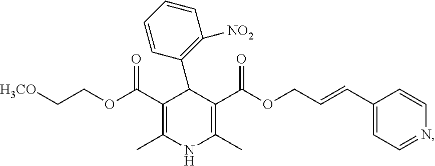 [SMILES: COCCOC(=O)C1=C(C)NC(C)=C(C(=O)OC/C=C/c2ccncc2)C1c1ccccc1[N+](=O)[O-]]

or a pharmacologically acceptable salt thereof or a solvate of them.
45. The method of embodiment 9, wherein the compound of Formula (I) is

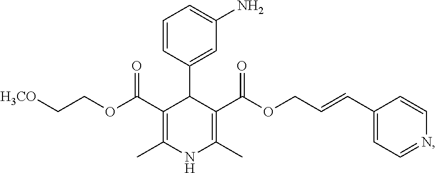 [SMILES: COCCOC(=O)C1=C(C)NC(C)=C(C(=O)OC/C=C/c2ccncc2)C1c1cccc(N)c1]

or a pharmacologically acceptable salt thereof or a solvate of them.
46. The method of embodiment 9, wherein the compound of Formula (I) is

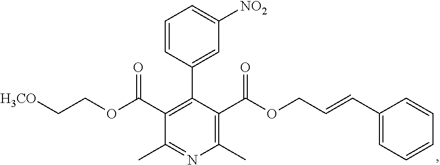 [SMILES: COCCOC(=O)c1c(C)nc(C)c(C(=O)OC/C=C/c2ccccc2)c1-c1cccc([N+](=O)[O-])c1]

or a pharmacologically acceptable salt thereof or a solvate of them.
47. The method of embodiment 9, wherein the compound of Formula (I) is

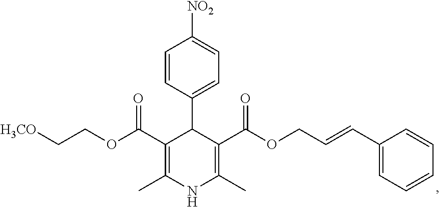 [SMILES: COCCOC(=O)C1=C(C)NC(C)=C(C(=O)OC/C=C/c2ccccc2)C1c1ccc([N+](=O)[O-])cc1]

or a pharmacologically acceptable salt thereof or a solvate of them.
48. The method of embodiment 9, wherein the compound of Formula (I) is

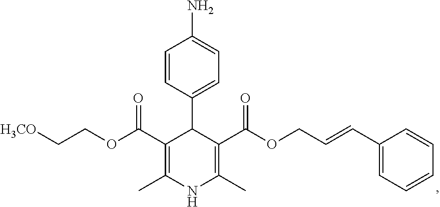 [SMILES: COCCOC(=O)C1=C(C)NC(C)=C(C(=O)OC/C=C/c2ccccc2)C1c1ccc(N)cc1]

or a pharmacologically acceptable salt thereof or a solvate of them.
49. The method of embodiment 9, wherein the compound of Formula (I) is

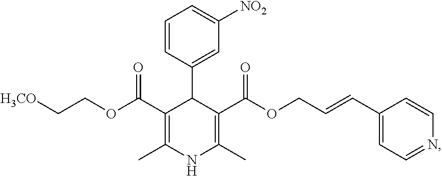 [SMILES: COCCOC(=O)C1=C(C)NC(C)=C(C(=O)OC/C=C/c2ccncc2)C1c1cccc([N+](=O)[O-])c1]

or a pharmacologically acceptable salt thereof or a solvate of them.
50. The method of embodiment 9, wherein the compound of Formula (I) is

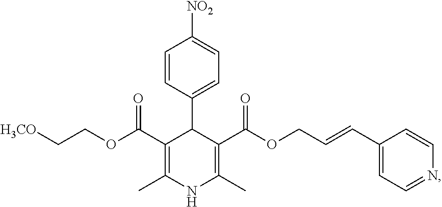 [SMILES: COCCOC(=O)C1=C(C)NC(C)=C(C(=O)OC/C=C/c2ccncc2)C1c1ccc([N+](=O)[O-])cc1]

or a pharmacologically acceptable salt thereof or a solvate of them.
51. The method of any one of embodiments 1 to 50, wherein the agent or combination of agents comprises metformin or a pharmacologically acceptable salt thereof or a solvate of them.
52. The method of any one of embodiments 1 to 51, wherein the agent or combination of agents comprises P110 or a pharmacologically acceptable salt thereof or a solvate of them.
53. The method of any one of embodiments 1 to 52, wherein the agent or combination of agents comprises mdivi-1 or a pharmacologically acceptable salt thereof or a solvate of them.
54. The method of any one of embodiments 1 to 53, wherein the agent or combination of agents comprises berberine or a pharmacologically acceptable salt thereof or a solvate of them.
55. The method of any one of embodiments 1 to 54, wherein the agent or combination of agents comprises an agent or a combination of agents that activate toll-like receptors (TLRs), Dectin-1, Mannose receptor, Scavenger receptor A, CD14, CD36, an opsonic receptor, an apoptotic body receptor, or a combination thereof.
56. The method of embodiment 55, wherein the agent or combination of agents comprises an agent that activates TLRs.
57. The method of embodiment 56, which comprises administering a combination of agents comprising two or more agents that activate TLRs.
58. The method of any one of embodiments 55 to 57, wherein the agent or combination of agents comprises an agent that activates Dectin-1.
59. The method of embodiment 58, which comprises administering a combination of agents comprising two or more agents that activate Dectin-1.
60. The method of any one of embodiments 55 to 59, wherein the agent or combination of agents comprises an agent that activates Mannose receptor.
61. The method of embodiment 60, which comprises administering a combination of agents comprising two or more agents that activate the Mannose receptor.
62. The method of any one of embodiments 55 to 61, wherein the agent or combination of agents comprises an agent that activates Scavenger receptor A.
63. The method of embodiment 62, which comprises administering a combination of agents comprising two or more agents that activate Scavenger receptor A.
64. The method of any one of embodiments 55 to 63, wherein the agent or combination of agents comprises an agent that activates CD14.
65. The method of embodiment 64, which comprises administering a combination of agents comprising two or more agents that activate CD14.
66. The method of any one of embodiments 55 to 65, wherein the agent or combination of agents comprises an agent that activates CD36.
67. The method of embodiment 66, which comprises administering a combination of agents comprising two or more agents that activate CD36.
68. The method of any one of embodiments 55 to 67, wherein the agent or combination of agents comprises an agent that activates an opsonic receptor.
69. The method of embodiment 68, which comprises administering a combination of agents comprising two or more agents that activate an opsonic receptor.
70. The method of embodiment 55, wherein the agent or combination of agents comprises an agent that activates an apoptotic body receptor.
71. The method of embodiment 70, which comprises administering a combination of agents comprising two or more agents that activate an apoptotic body receptor.
72. The method of any one of embodiments 55 to 71, wherein the agent or combination of agents comprises 1,3-beta glucan, Mannan, Lipopolysaccharide, lipoteichoic acid, Lipopolysaccharide-binding protein, Plasmodium falciparum-infected erythrocytes, IgG, IgA, IgE or Phosphatidylserine.
73. The method of any one of embodiments 1 to 72, wherein the agent or combination of agents comprises an agent that activates PGC1α.
74. The method of embodiment 73, which comprises administering a combination of agents comprising two or more agents that activate PGC1α.
75. The method of embodiment 73 or embodiment 74, wherein the agent or combination of agents comprises metformin or a pharmacologically acceptable salt thereof or a solvate of them.
76. The method of any one of embodiments 1 to 75, wherein the agent or combination of agents comprises an agent that inhibits the PI3K-AKT-mTOR pathway.
77. The method of embodiment 76, wherein the agent or combination of agents comprises a PI3K inhibitor.
78. The method of embodiment 77, which comprises administering a combination of agents comprising two or more PIK3 inhibitors.
79. The method of any one of embodiments 76 to 78, wherein the agent or combination of agents comprises an AKT inhibitor.
80. The method of embodiment 79, which comprises administering a combination of agents comprising two or more AKT inhibitors.
81. The method of any one of embodiments 76 to 80, wherein the agent or combination of agents comprises a mTOR inhibitor.
82. The method of embodiment 81, which comprises administering a combination of agents comprising two or more mTOR inhibitors.
83. The method of any one of embodiments 76 to 82, wherein the agent or combination of agents comprises rapamycin or a pharmacologically acceptable salt thereof or a solvate of them.
84. The method of any one of embodiments 1 to 83, wherein the agent or combination of agents comprises one or more lactic acid releasing bacteria species.
85. The method of embodiment 84, wherein the agent or combination of agents comprises a Bifidobacterium and/or Lactobacillus acidophilus.
86. The method of any one of embodiments 1 to 85, wherein the agent or combination of agents comprises a component of or a combination of components of a cell free extract from a lactic acid releasing bacteria culture medium.
87. The method of embodiment 86, wherein the lactic acid releasing bacteria comprises a Bifidobacterium and/or Lactobacillus acidophilus.
88. The method of any one of embodiments 1 to 87, wherein the agent or one or more agents in the combination of agents is/are formulated with a bile acid derivative.
89. The method of embodiment 88, wherein the bile acid derivative is ursodeoxycholic acid or a pharmacologically acceptable salt thereof or a solvate of them.
90. The method of any one of embodiments 1 to 89, wherein the disease is cancer, which optionally has a KRAS mutation.
91. The method of embodiment 90, wherein the cancer is a hematological cancer.
92. The method of embodiment 90, wherein the cancer is a solid tumor.
93. The method of embodiment 90, wherein the cancer is leukemia.
94. The method of embodiment 90, wherein the cancer is lymphoma.
95. The method of embodiment 90, wherein the cancer is myeloma.
96. The method of embodiment 90, wherein the cancer is multiple myeloma.
97. The method of embodiment 90, wherein the cancer is acute myeloid leukemia.
98. The method of embodiment 90, wherein the cancer is acute lymphocytic leukemia.
99. The method of embodiment 90, wherein the cancer is non-Hodgkin lymphoma.
100. The method of embodiment 90, wherein the cancer is diffuse large B-Cell lymphoma.
101. The method of embodiment 90, wherein the cancer is melanoma, optionally wherein the melanoma has a BRAF mutation.
102. The method of embodiment 90, wherein the cancer is leiomyosarcoma.
103. The method of embodiment 90, wherein the cancer is breast cancer.
104. The method of embodiment 90, wherein the cancer is liver cancer.
105. The method of embodiment 90, wherein the cancer is colorectal cancer.
106. The method of embodiment 90, wherein the cancer is pancreatic cancer, optionally wherein the pancreatic cancer is pancreatic ductal adenocarcinoma.
107. The method of embodiment 90, wherein the cancer is lung cancer, which is optionally small cell lung cancer or non-small cell lung cancer, optionally lung adenocarcinoma.
108. The method of embodiment 90, wherein the cancer is osteosarcoma.
109. The method of embodiment 90, wherein the cancer is head and neck cancer.
110. The method of any one of embodiments 90 to 109, when depending directly or indirectly from embodiment 3, which comprises administering a mitochondrial fission inhibitor as monotherapy for the cancer.
111. The method of any one of embodiments 90 to 109, wherein the subject is administered one or more additional agents.
112. The method of embodiment 111, wherein the one or more additional agents comprise a CD47 inhibitor and/or SIRPα inhibitor.
113. The method of embodiment 112, wherein the one or more additional agents comprise a CD47 inhibitor.
114. The method of embodiment 113, wherein the CD47 inhibitor comprises an antibody or antigen-binding fragment thereof.
115. The method of embodiment 113, wherein the CD47 inhibitor comprises a SIRPα-Fc fusion protein.
116. The method of embodiment 113, wherein the CD47 inhibitor comprises a SIRPα variant protein.
117. The method of embodiment 113, wherein the CD47 inhibitor is Magrolimab.
118. The method of embodiment 113, wherein the CD47 inhibitor is CC-90002.
119. The method of embodiment 113, wherein the CD47 inhibitor is AO-176.
120. The method of embodiment 113, wherein the CD47 inhibitor is IBI-188.
121. The method of embodiment 113, wherein the CD47 inhibitor is SHR-1063.
122. The method of embodiment 113, wherein the CD47 inhibitor is AMMS4-G4
123. The method of embodiment 113, wherein the CD47 inhibitor is SIRPα-Fc fusion protein.
124. The method of embodiment 113, wherein the CD47 inhibitor is TTI-621.
125. The method of embodiment 113, wherein the CD47 inhibitor is ALX148.
126. The method of embodiment 113, wherein the CD47 inhibitor is CV1.
127. The method of any one of embodiments 112 to 126, wherein the one or more additional agents comprise a SIRPα inhibitor.
128. The method of embodiment 127, wherein the SIRPα inhibitor comprises an antibody or antigen-binding fragment thereof.
129. The method of embodiment 127, wherein the SIRPα inhibitor is KWAR23.
130. The method of embodiment 127, wherein the SIRPα inhibitor is CC-95251.
131. The method of embodiment 127, wherein the SIRPα inhibitor is BI 765063.
132. The method of embodiment 127, wherein the SIRPα inhibitor comprises a soluble CD47 peptide.
133. The method of any one of embodiments 111 to 132, wherein the one or more additional agents comprises a standard of care therapy for the cancer.
134. The method of any one of embodiments 111 to 133, wherein when the cancer is pancreatic cancer, the one or more additional agents comprises gemcitabine and/or pacritaxel.
135. The method of any one of embodiments 111 to 133, wherein when the cancer is lung adenocarcinoma, the one or more additional agents comprises cisplatin and/or carboplatin.
136. The method of any one of embodiments 111 to 133, wherein when the cancer is colorectal cancer, the one or more additional agents comprises cetuximab and/or panitumumab.
137. The method of any one of embodiments 111 to 136, wherein the one or more additional agents comprises an anti-PD1 antibody and/or an anti-PD-L1 antibody.
138. The method of embodiment 137, wherein the one or more additional agents comprises an anti-PD1 antibody.
139. The method of embodiment 138, wherein the anti-PD1 antibody is cemiplimab.
140. The method of embodiment 138, wherein the anti-PD1 antibody is nivolumab.
141. The method of embodiment 138, wherein the anti-PD1 antibody is pembrolizumab.
142. The method of any one of embodiments 137 to 141, wherein the one or more additional agents comprises an anti-PD-L1 antibody.
143. The method of embodiment 142, wherein the anti-PD-L1 antibody is avelumab.
144. The method of embodiment 142, wherein the anti-PD-L1 antibody is durvalumab.
145. The method of embodiment 142, wherein the anti-PD-L1 antibody is atezolizumab.
146. The method of any one of embodiments 111 to 145, further comprising administering the one or more additional agents to the subject.
147. The method of any one of embodiments 111 to 146, wherein one or more of the one or more additional agents is/are formulated with a bile acid derivative.
148. The method of embodiment 147, wherein the bile acid derivative is ursodeoxycholic acid or a pharmacologically acceptable salt thereof or a solvate of them.
149. The method of any one of embodiments 1 to 89, when depending directly or indirectly from embodiment 1, wherein the phagocytosis deficiency-related disease or condition is an infectious disease, a neurodegenerative disease, inflammation, an inflammatory disease, or a lysosomal disease.
150. The method of embodiment 149, wherein the disease or condition is an infectious disease.
151. The method of embodiment 150, wherein the infectious disease is caused by a bacterium.
152. The method of embodiment 150, wherein the infectious disease is caused by a virus.
153. The method of embodiment 149, wherein the disease or condition is a neurodegenerative disease.
154. The method of embodiment 153, wherein the neurodegenerative disease is Alzheimer's disease (AD) or other dementia, Parkinson's disease (PD), Nasu-Hakola disease, prion disease, amyotrophic lateral sclerosis (ALS), Friedreich's ataxia, Huntington's disease, Lewy body disease, spinal muscular atrophy, progressive supranuclear palsy (PSP), or adrenoleukodystrophy (ALD) .
155. The method of embodiment 154, wherein the neurodegenerative disease is Alzheimer's disease (AD).
156. The method of embodiment 154, wherein the neurodegenerative disease is dementia.
157. The method of embodiment 154, wherein the neurodegenerative disease is Parkinson's disease (PD).
158. The method of embodiment 154, wherein the neurodegenerative disease is Nasu-Hakola disease.
159. The method of embodiment 154, wherein the neurodegenerative disease is prion disease.
160. The method of embodiment 154, wherein the neurodegenerative disease is amyotrophic lateral sclerosis (ALS).
161. The method of embodiment 154, wherein the neurodegenerative disease is Friedreich's ataxia.
162. The method of embodiment 154, wherein the neurodegenerative disease is Huntington's disease.
163. The method of embodiment 154, wherein the neurodegenerative disease is Lewy body disease.
164. The method of embodiment 154, wherein the neurodegenerative disease is spinal muscular atrophy.
165. The method of embodiment 154, wherein the neurodegenerative disease is progressive supranuclear palsy (PSP).
166. The method of embodiment 154, wherein the neurodegenerative disease is adrenoleukodystrophy (ALD).
167. The method of embodiment 149, wherein the disease or condition is inflammation.
168. The method of embodiment 149, wherein the disease is an inflammatory disease.
169. The method of embodiment 168, wherein the inflammatory disease is an autoimmune disease.
170. The method of embodiment 169, wherein the autoimmune disease is systemic lupus erythematosus (SLE), rheumatoid arthritis (RA), or autoimmune lymphoproliferative syndrome (ALPS).
171. The method of embodiment 170, wherein the autoimmune disease is SLE.
172. The method of embodiment 170, wherein the autoimmune disease is RA.
173. The method of embodiment 170, wherein the autoimmune disease is ALPS.
174. The method of embodiment 149, wherein the disease is a lysosomal storage disease.
175. The method of embodiment 174, wherein the lysosomal storage disease is Gaucher disease, Fabry disease, Niemann-Pick disease, Hunter syndrome, Glycogen storage disease II (Pompe disease), or Tay-Sachs disease.
176. The method of embodiment 175, wherein the lysosomal storage disease is Gaucher disease.
177. The method of embodiment 175, wherein the lysosomal storage disease is Fabry disease.
178. The method of embodiment 175, wherein the lysosomal storage disease is Niemann-Pick disease.
179. The method of embodiment 175, wherein the lysosomal storage disease is Hunter syndrome.
180. The method of embodiment 175, wherein the lysosomal storage disease is Glycogen storage disease II (Pompe disease).
181. The method of embodiment 175, wherein the lysosomal storage disease is Tay-Sachs disease.
182. The method of any one of embodiments 1 to 89, when depending directly or indirectly from embodiment 1, wherein the phagocytosis deficiency-related disease or condition is a musculoskeletal degenerative disease.
183. The method of embodiment 182, wherein the musculoskeletal degenerative disease is a muscular dystrophy.
184. The method of embodiment 183, wherein the muscular dystrophy is Duchenne muscular dystrophy (DMD).
185. The method of any one of embodiments 1 to 184, wherein the subject has phagocytic deficiency.
186. A method of treating a subject having Duchenne muscular dystrophy (DMD), comprising administering to the subject a therapeutically effective amount of cilnidipine or a pharmacologically acceptable salt thereof or a solvate of them.
187. A combination comprising one or more phagocytosis activating agents and a CD47 and/or a SIRPα inhibitor, optionally wherein the one or more phagocytosis activating agents comprise one or more phagocytosis activating agents described in embodiments 1 to 89, and optionally wherein the CD47 and/or SIRPα inhibitor is any CD47 or SIRPα inhibitor described in any of embodiments 112 to 132.
188. The combination of embodiment 187 for use in treating a phagocytosis deficiency-related disease or condition.
189. The combination for use according to embodiment 188, wherein the phagocytosis deficiency-related disease or condition is a cancer, optionally wherein the cancer is a cancer as described in any one of embodiments 90 to 109.
190. A mitochondrial fission inhibitor or combination of mitochondrial fission inhibitors for use in treating cancer, optionally wherein the mitochondrial fission inhibitor or combination of mitochondrial fission inhibitors comprise a mitochondrial fission inhibitor or combination of mitochondrial fission inhibitors as described in any one of embodiments 3 to 54, optionally wherein the cancer is a cancer as described in any one of embodiments 90 to 109.
191. The mitochondrial fission inhibitor or combination of mitochondrial fission inhibitors for use according to embodiment 190, for use as monotherapy.
192. The mitochondrial fission inhibitor or combination of mitochondrial fission inhibitors for use according to embodiment 191, for use in combination with one or more additional agents.
193. The mitochondrial fission inhibitor or combination of mitochondrial fission inhibitors for use according to embodiment 192, wherein the one or more additional agents comprise one or more agents as described in any of embodiments 55 to 89 and 111 to 148.
194. A mitochondrial fission inhibitor or combination of mitochondrial fission inhibitors for use in treating a phagocytosis deficiency-related disease or condition, optionally wherein the mitochondrial fission inhibitor or combination of mitochondrial fission inhibitors comprises a mitochondrial fission inhibitor or combination of mitochondrial fission inhibitors as described in any one of embodiments 3 to 54.
195. The mitochondrial fission inhibitor or combination of mitochondrial fission inhibitors for use according to embodiment 194, for use in combination with one or more additional agents.
196. The mitochondrial fission inhibitor or combination of mitochondrial fission inhibitors for use according to embodiment 195, wherein the one or more additional agents comprise one or more agents as described in any of embodiments 55 to 89.
197. The mitochondrial fission inhibitor for use according to any one of embodiments 194 to 196, wherein the phagocytosis deficiency related disease or condition is a disease or condition described in any one of embodiments 149 to 184.
198. An agent or combination of agents for use in a method of treating a subject having a phagocytosis deficiency-related disease or condition, wherein the agent or combination of agents comprises a phagocytosis activating agent, optionally wherein:
   (a) the phagocytosis deficiency-related disease or condition is a cancer, optionally wherein the cancer is pancreatic cancer; and/or
   (b) the agent or combination of agents comprises cilnidipine or a pharmacologically acceptable salt thereof or a solvate of them.
199. The agent or combination of agents for use according to embodiment 198, wherein the agent or combination of agents comprises a mitochondrial fission inhibitor.
200. The agent or combination of agents for use according to embodiment 198 or embodiment 199, wherein the agent or combination of agents comprises a Drp1 inhibitor.
201. The agent or combination of agents for use according to any one of embodiments 198 to 200, wherein the agent or combination of agents comprises cilnidipine, P110, metformin, mdivi-1, berberine, a pharmacologically acceptable salt thereof or a solvate of them or a combination of any of the foregoing.
202. The agent or combination of agents for use according to any one of embodiments 198 to 201, wherein the agent or combination of agents comprises cilnidipine or a pharmacologically acceptable salt thereof or a solvate of them.
203. The agent or combination of agents for use according to any one of embodiments 198 to 201, wherein the agent or combination of agents comprises a compound of Formula (I):

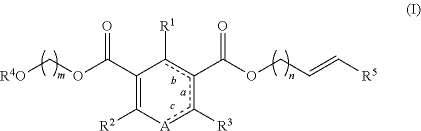

or a pharmacologically acceptable salt thereof or a solvate of them, wherein:
R¹ is phenyl substituted with one to three substituents each of which is independently NO₂, NH₂, OH, C₁-C₆ alkyl, C₁-C₆ haloalkyl, or C₁-C₆ alkoxyalkyl, provided that at least one substituent is NO₂ or NH₂;
R² is H, C₁-C₆ alkyl, C₁-C₆ haloalkyl, or C₁-C₆ alkoxyalkyl;
R³ is H, C₁-C₆ alkyl, C₁-C₆ haloalkyl, or C₁-C₆ alkoxyalkyl;
R⁴ is C₁-C₆ alkyl or C₁-C₆ haloalkyl;
R5 is phenyl or pyridinyl, wherein the phenyl or pyridinyl is unsubstituted or substituted with one to three substituents each of which is independently NO₂, NH₂, OH, C₁-C₆ alkyl, C₁-C₆ haloalkyl, or C₁-C₆ alkoxyalkyl;
either (i) bond a is present and bonds b and c are absent or (ii) bonds b and c are present and bond a is absent;
A is NH when bond a is present and N when bonds b and c are present;
m is an integer from 1 to 4; and
n is an integer from 1 to 3,
optionally wherein the compound of Formula (I) is not cilnidipine.
204. The agent or combination of agents for use according to any one of embodiments 198 to 203, wherein the agent or combination of agents comprises an agent or a combination of agents that activate toll-like receptors (TLRs), Dectin-1, Mannose receptor, Scavenger receptor A, CD14, CD36, an opsonic receptor, an apoptotic body receptor, or a combination thereof.
205. The agent or combination of agents for use according to embodiment 204, wherein the agent or combination of agents comprises 1,3-beta glucan, Mannan, Lipopolysaccharide, lipoteichoic acid, Lipopolysaccharide-binding protein, Plasmodium falciparum-infected erythrocytes, IgG, IgA, IgE or Phosphatidylserine.
206. The agent or combination of agents for use according to any one of embodiments 198 to 205, wherein the agent or combination of agents comprises an agent that activates PGC1α.
207. The agent or combination of agents for use according to embodiment 206, wherein the agent or combination of agents comprises metformin or a pharmacologically acceptable salt thereof or a solvate of them.
208. The agent or combination of agents for use according to any one of embodiments 198 to 207, wherein the agent or combination of agents comprises an agent that inhibits the PI3K-AKT-mTOR pathway.
209. The agent or combination of agents for use according to embodiment 208, wherein the agent or combination of agents comprises (i) a PI3K inhibitor, (ii) an AKT inhibitor, (iii) a mTOR inhibitor, (iv) rapamycin or a pharmacologically acceptable salt thereof or a solvate of them, or (v) a combination of the foregoing.
210. The agent or combination of agents for use according to any one of embodiments 198 to 209, wherein the agent or combination of agents comprises one or more lactic acid releasing bacteria species, optionally wherein the agent or combination of agents comprises a Bifidobacterium and/or Lactobacillus acidophilus.
211. The agent or combination of agents for use according to any one of embodiments 198 to 210, wherein the agent or combination of agents comprises a component of or a combination of components of a cell free extract from a lactic acid releasing bacteria culture medium, optionally wherein the lactic acid releasing bacteria comprises a Bifidobacterium and/or Lactobacillus acidophilus.
212. The agent or combination of agents for use according to any one of embodiments 198 to 211, wherein the agent or one or more agents in the combination of agents is/are formulated with a bile acid derivative, optionally wherein the bile acid derivative is ursodeoxycholic acid or a pharmacologically acceptable salt thereof or a solvate of them.
213. The agent or combination of agents for use according to any one of embodiments 198 to 212, wherein the phagocytosis deficiency-related disease or condition is cancer, which optionally has a KRAS mutation, optionally wherein the cancer is:
   (a) pancreatic cancer;
   (b) a hematological cancer;
   (c) a solid tumor;
   (d) leukemia;
   (e) lymphoma;
   (f) myeloma;
   (g) multiple myeloma;
   (h) acute myeloid leukemia;
   (i) acute lymphocytic leukemia;
   (j) non-Hodgkin lymphoma;
   (k) diffuse large B-Cell lymphoma;
   (l) melanoma, optionally wherein the melanoma has a BRAF mutation;
   (m) leiomyosarcoma;
   (n) breast cancer;
   (o) liver cancer;
   (p) colorectal cancer;
   (q) lung cancer, which is optionally small cell lung cancer or non-small cell lung cancer, optionally lung adenocarcinoma;
   (r) osteosarcoma; or
   (s) head and neck cancer.
214. The agent or combination of agents for use according to embodiment 213, wherein the cancer is pancreatic cancer, optionally wherein the pancreatic cancer is pancreatic ductal adenocarcinoma.
215. The agent for use according to embodiment 213 or embodiment 214, which is a mitochondrial fission inhibitor and wherein the method comprises administering the mitochondrial fission inhibitor as monotherapy for the cancer.
216. The agent or combination of agents for use according to embodiments 213 or embodiment 214, wherein the method comprises administering one or more additional agents to the subject.
217. The agent or combination of agents for use according to embodiment 216, wherein the one or more additional agents comprise a CD47 inhibitor and/or SIRPα inhibitor.
218. The agent or combination of agents for use according to embodiment 217, wherein the one or more additional agents comprise a CD47 inhibitor, optionally wherein the CD47 inhibitor is:
   (a) an antibody or antigen-binding fragment thereof;
   (b) a SIRPα-Fc fusion protein;
   (c) a SIRPα variant protein;
   (d) Magrolimab;
   (e) CC-90002;
   (f) AO-176;
   (g) IBI-188;
   (h) SHR-1063;
   (i) AMMS4-G4;
   (j) TTI-621;
   (k) ALX148; or
   (l) CV1.
219. The agent or combination of agents for use according to embodiment 217 or embodiment 218, wherein the one or more additional agents comprise a SIRPα inhibitor, optionally wherein the SIRPα inhibitor is:
   (a) an antibody or antigen-binding fragment thereof;
   (b) KWAR23;
   (c) CC-95251;
   (d) BI 765063; or
   (e) a soluble CD47 peptide.
220. The agent or combination of agents for use according to any one of embodiments 216 to 219, wherein the one or more additional agents comprises a standard of care therapy for the cancer.
221. The agent or combination of agents for use according to any one of embodiments 216 to 220, wherein when the cancer is pancreatic cancer, and the one or more additional agents comprises gemcitabine and/or pacritaxel.
222. The agent or combination of agents for use according to embodiment 221, wherein when the cancer is pancreatic cancer, and the one or more additional agents comprises gemcitabine.
223. The agent or combination of agents for use according to any one of embodiments 216 to 220, wherein when the cancer is lung adenocarcinoma, and the one or more additional agents comprises cisplatin and/or carboplatin.
224. The agent or combination of agents for use according to any one of embodiments 216 to 220, wherein when the cancer is colorectal cancer, and the one or more additional agents comprises cetuximab and/or panitumumab.
225. The agent or combination of agents for use according to any one of embodiments 216 to 224, wherein the one or more additional agents comprises an anti-PD1 antibody, optionally wherein the anti-PD1 antibody is cemiplimab, nivolumab, or pembrolizumab, and/or an anti-PD-L1 antibody, optionally wherein the anti-PD-L1 antibody is avelumab, durvalumab, or atezolizumab.
226. The agent or combination of agents for use according to any one of embodiments 216 to 225, further comprising administering the one or more additional agents to the subject.
227. The agent or combination of agents for use according to any one of embodiments 216 to 226, wherein one or more of the one or more additional agents is/are formulated with a bile acid derivative, optionally wherein the bile acid derivative is ursodeoxycholic acid or a pharmacologically acceptable salt thereof or a solvate of them.
228. The agent or combination of agents for use according to any one of embodiments 198 to 212, wherein the phagocytosis deficiency-related disease or condition is:
   (a) a musculoskeletal degenerative disease, which is optionally a muscular dystrophy such as Duchenne muscular dystrophy (DMD);
   (b) an infectious disease, optionally caused by a bacterium or a virus;
   (c) a neurodegenerative disease, which is optionally Alzheimer's disease (AD) or other dementia, Parkinson's disease (PD), Nasu-Hakola disease, prion disease, amyotrophic lateral sclerosis (ALS), Friedreich's ataxia, Huntington's disease, Lewy body disease, spinal muscular atrophy, progressive supranuclear palsy (PSP), or adrenoleukodystrophy (ALD);
   (d) inflammation;
   (e) an inflammatory disease, which is optionally an autoimmune disease, such as systemic lupus erythematosus (SLE), rheumatoid arthritis (RA), or autoimmune lymphoproliferative syndrome (ALPS); or
   (f) a lysosomal storage disease, which is optionally Gaucher disease, Fabry disease, Niemann-Pick disease, Hunter syndrome, Glycogen storage disease II (Pompe disease), or Tay-Sachs disease.
229. The agent or combination of agents for use according to embodiment 228, wherein the phagocytosis deficiency-related disease or condition is Duchenne muscular dystrophy (DMD).
230. The agent or combination of agents for use according to any one of embodiments 198 to 229, wherein the subject has phagocytic deficiency.
231. An agent which is cilnidipine or a pharmacologically acceptable salt thereof or a solvate of them for use in a method of treating a subject having pancreatic cancer, optionally wherein the pancreatic cancer is pancreatic ductal adenocarcinoma.
232. The agent for use according to embodiment 231, wherein the agent is for use as monotherapy.
233. The agent for use according to embodiment 232, wherein the method comprises administering an amount of the agent to the subject effective to slow growth of a tumor in the subject.
234. The agent for use according to embodiment 231, wherein the agent is for use in combination with gemcitabine.
235. The agent for use according to embodiment 234, wherein the method comprises administering an amount of the agent and an amount of the gemcitabine to the subject which together are effective to slow growth of a tumor in the subject.
236. An agent which is cilnidipine or a pharmacologically acceptable salt thereof or a solvate of them for use in a method of treating a subject having Duchenne muscular dystrophy (DMD).
237. The agent for use according to embodiment 236, wherein the method comprises administering an amount of the agent effect to reduce creatinine kinase (CK) and/or lactose dehydrogenase (LDH) in the subject's plasma.
238. A pharmaceutical composition comprising phagocytes having enhanced phagocytic activity.
239. The pharmaceutical composition of embodiment 238, which comprises phagocytes that have been contacted with one or more mitochondrial fission inhibitors ex vivo.
240. The pharmaceutical composition of embodiment 239, wherein the one or more mitochondrial fission inhibitors comprise a mitochondrial fission inhibitor or combination of mitochondrial fission inhibitors as described in any one of embodiments 3 to 54.
241. The pharmaceutical composition of embodiment 239 or embodiment 240, wherein the one or more mitochondrial fission inhibitors comprise cilnidipine or a pharmacologically acceptable salt thereof or a solvate of them.
242. The pharmaceutical composition of any one of embodiments 238 to 241, wherein the phagocytes comprises monocytes, macrophages, neutrophils, dendritic cells, mast cells, or any combination thereof.
243. The pharmaceutical composition of embodiment 242, wherein the phagocytes comprise monocytes.
244. The pharmaceutical composition of embodiment 242 or embodiment 243, wherein the phagocytes comprise macrophages.
245. The pharmaceutical composition of any one of embodiments 242 to 244, wherein the phagocytes comprise neutrophils.
246. The pharmaceutical composition of any one of embodiments 242 to 245, wherein the phagocytes comprise dendritic cells.
247. The pharmaceutical composition of any one of embodiments 242 to 246, wherein the phagocytes comprise mast cells.
248. The pharmaceutical composition of any one of embodiments 238 to 247, for use in a method of treating a subject having a phagocytosis deficiency-related disease or condition.
249. The pharmaceutical composition for use according to embodiment 248, wherein the phagocytosis deficiency-related disease or condition is a disease or condition described in any one of embodiments 90 to 109 and 149 to 185.
250. The pharmaceutical composition for use according to embodiment 249, wherein the disease or condition is a cancer.
251. The pharmaceutical composition for use according to any one of embodiments 248 to 250, wherein the phagocytes are autologous to the subject.
252. The pharmaceutical composition for use according to any one of embodiments 248 to 250, wherein the phagocytes are allogeneic to the subject.
253. A process for making the pharmaceutical composition of any one of embodiments 239 to 252, comprising contacting the phagocytes with the one or more mitochondrial fission inhibitors ex vivo.
254. A mitochondrial fission inhibitor for use in preparing the pharmaceutical composition of any one of embodiments 239 to 252, optionally wherein the mitochondrial fission inhibitor comprises a mitochondrial fission inhibitor as described in any one of embodiments 3 to 54.
255. The mitochondrial fission inhibitor for use according to embodiment 254, which is cilnidipine or a pharmacologically acceptable salt thereof or a solvate of them.

### 8. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

What is claimed is:

1. An agent or combination of agents for use in a method of treating a subject having a phagocytosis deficiency-related disease or condition, wherein the agent or combination of agents comprises a phagocytosis activating agent, optionally wherein:
(a) the phagocytosis deficiency-related disease or condition is a cancer, optionally wherein the cancer is pancreatic cancer; and/or
(b) the agent or combination of agents comprises cilnidipine or a pharmacologically acceptable salt thereof or a solvate of them.

2. The agent or combination of agents for use according to claim 1, wherein the agent or combination of agents comprises a mitochondrial fission inhibitor.

3. The agent or combination of agents for use according to claim 1 or claim 2, wherein the agent or combination of agents comprises a Drp1 inhibitor.

4. The agent or combination of agents for use according to any one of claims 1 to 3, wherein the agent or combination of agents comprises cilnidipine, P110, metformin, mdivi-1, berberine, a pharmacologically acceptable salt thereof or a solvate of them or a combination of any of the foregoing.

5. The agent or combination of agents for use according to any one of claims 1 to 4, wherein the agent or combination of agents comprises cilnidipine or a pharmacologically acceptable salt thereof or a solvate of them.

6. The agent or combination of agents for use according to any one of claims 1 to 5, wherein the agent or combination of agents comprises a compound of Formula (I): 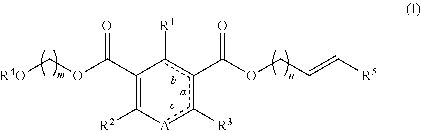
or a pharmacologically acceptable salt thereof or a solvate of them, wherein:
R¹ is phenyl substituted with one to three substituents each of which is independently NO₂, NH₂, OH, C₁-C₆ alkyl, C₁-C₆ haloalkyl, or C₁-C₆ alkoxyalkyl, provided that at least one substituent is NO₂ or NH₂;
R² is H, C₁-C₆ alkyl, C₁-C₆ haloalkyl, or C₁-C₆ alkoxyalkyl;
R³ is H, C₁-C₆ alkyl, C₁-C₆ haloalkyl, or C₁-C₆ alkoxyalkyl;
R⁴ is C₁-C₆ alkyl or C₁-C₆ haloalkyl;
R⁵ is phenyl or pyridinyl, wherein the phenyl or pyridinyl is unsubstituted or substituted with one to three substituents each of which is independently NO₂, NH₂, OH, C₁-C₆ alkyl, C₁-C₆ haloalkyl, or C₁-C₆ alkoxyalkyl;
either (i) bond a is present and bonds b and c are absent or (ii) bonds b and c are present and bond a is absent;
A is NH when bond a is present and N when bonds b and c are present;
m is an integer from 1 to 4; and
n is an integer from 1 to 3,
optionally wherein the compound of Formula (I) is not cilnidipine.

7. The agent or combination of agents for use according to any one of claims 1 to 6, wherein the agent or combination of agents comprises an agent or a combination of agents that activate toll-like receptors (TLRs), Dectin-1, Mannose receptor, Scavenger receptor A, CD14, CD36, an opsonic receptor, an apoptotic body receptor, or a combination thereof.

8. The agent or combination of agents for use according to claim 7, wherein the agent or combination of agents comprises 1,3-beta glucan, Mannan, Lipopolysaccharide, lipoteichoic acid, Lipopolysaccharide-binding protein, Plasmodium falciparum-infected erythrocytes, IgG, IgA, IgE or Phosphatidylserine.

9. The agent or combination of agents for use according to any one of claims 1 to 8, wherein the agent or combination of agents comprises an agent that activates PGC1α.

10. The agent or combination of agents for use according to claim 9, wherein the agent or combination of agents comprises metformin or a pharmacologically acceptable salt thereof or a solvate of them.

11. The agent or combination of agents for use according to any one of claims 1 to 10, wherein the agent or combination of agents comprises an agent that inhibits the PI3K-AKT-mTOR pathway.

12. The agent or combination of agents for use according to claim 11, wherein the agent or combination of agents comprises (i) a PI3K inhibitor, (ii) an AKT inhibitor, (iii) a mTOR inhibitor, (iv) rapamycin or a pharmacologically acceptable salt thereof or a solvate of them, or (v) a combination of the foregoing.

13. The agent or combination of agents for use according to any one of claims 1 to 12, wherein the agent or combination of agents comprises one or more lactic acid releasing bacteria species, optionally wherein the agent or combination of agents comprises a Bifidobacterium and/or Lactobacillus acidophilus.

14. The agent or combination of agents for use according to any one of claims 1 to 13, wherein the agent or combination of agents comprises a component of or a combination of components of a cell free extract from a lactic acid releasing bacteria culture medium, optionally wherein the lactic acid releasing bacteria comprises a Bifidobacterium and/or Lactobacillus acidophilus.

15. The agent or combination of agents for use according to any one of claims 1 to 14, wherein the agent or one or more agents in the combination of agents is/are formulated with a bile acid derivative, optionally wherein the bile acid derivative is ursodeoxycholic acid or a pharmacologically acceptable salt thereof or a solvate of them.

16. The agent or combination of agents for use according to any one of claims 1 to 15, wherein the phagocytosis deficiency-related disease or condition is cancer, which optionally has a KRAS mutation, optionally wherein the cancer is:
(a) pancreatic cancer;
(b) a hematological cancer;
(c) a solid tumor;
(d) leukemia;
(e) lymphoma;
(f) myeloma;
(g) multiple myeloma;
(h) acute myeloid leukemia;
(i) acute lymphocytic leukemia;
(j) non-Hodgkin lymphoma;
(k) diffuse large B-Cell lymphoma;
(l) melanoma, optionally wherein the melanoma has a BRAF mutation;
(m) leiomyosarcoma;
(n) breast cancer;
(o) liver cancer;
(p) colorectal cancer;
(q) lung cancer, which is optionally small cell lung cancer or non-small cell lung cancer, optionally lung adenocarcinoma;
(r) osteosarcoma; or
(s) head and neck cancer.

17. The agent or combination of agents for use according to claim 16, wherein the cancer is pancreatic cancer, optionally wherein the pancreatic cancer is pancreatic ductal adenocarcinoma.

18. The agent for use according to claim 16 or claim 17, which is a mitochondrial fission inhibitor and wherein the method comprises administering the mitochondrial fission inhibitor as monotherapy for the cancer.

19. The agent or combination of agents for use according to claims 16 or claim 17, wherein the method comprises administering one or more additional agents to the subject.

20. The agent or combination of agents for use according to claim 19, wherein the one or more additional agents comprise a CD47 inhibitor and/or SIRPα inhibitor.

21. The agent or combination of agents for use according to claim 20, wherein the one or more additional agents comprise a CD47 inhibitor, optionally wherein the CD47 inhibitor is:
(a) an antibody or antigen-binding fragment thereof;
(b) a SIRPα-Fc fusion protein;
(c) a SIRPα variant protein;
(d) Magrolimab;
(e) CC-90002;
(f) AO-176;
(g) IBI-188;
(h) SHR-1063;
(i) AMMS4-G4;
(j) TTI-621;
(k) ALX148; or
(l) CV1.

22. The agent or combination of agents for use according to claim 20 or claim 21, wherein the one or more additional agents comprise a SIRPα inhibitor, optionally wherein the SIRPα inhibitor is:
(a) an antibody or antigen-binding fragment thereof;
(b) KWAR23;
(c) CC-95251;
(d) BI 765063; or
(e) a soluble CD47 peptide.

23. The agent or combination of agents for use according to any one of claims 19 to 22, wherein the one or more additional agents comprises a standard of care therapy for the cancer.

24. The agent or combination of agents for use according to any one of claims 19 to 23, wherein when the cancer is pancreatic cancer, and the one or more additional agents comprises gemcitabine and/or pacritaxel.

25. The agent or combination of agents for use according to claim 24, wherein when the cancer is pancreatic cancer, and the one or more additional agents comprises gemcitabine.

26. The agent or combination of agents for use according to any one of claims 19 to 23, wherein when the cancer is lung adenocarcinoma, and the one or more additional agents comprises cisplatin and/or carboplatin.

27. The agent or combination of agents for use according to any one of claims 19 to 23, wherein when the cancer is colorectal cancer, and the one or more additional agents comprises cetuximab and/or panitumumab.

28. The agent or combination of agents for use according to any one of claims 19 to 27, wherein the one or more additional agents comprises an anti-PD1 antibody, optionally wherein the anti-PD1 antibody is cemiplimab, nivolumab, or pembrolizumab, and/or an anti-PD-L1 antibody, optionally wherein the anti-PD-L1 antibody is avelumab, durvalumab, or atezolizumab.

29. The agent or combination of agents for use according to any one of claims 19 to 28, further comprising administering the one or more additional agents to the subject.

30. The agent or combination of agents for use according to any one of claims 19 to 29, wherein one or more of the one or more additional agents is/are formulated with a bile acid derivative, optionally wherein the bile acid derivative is ursodeoxycholic acid or a pharmacologically acceptable salt thereof or a solvate of them.

31. The agent or combination of agents for use according to any one of claims 1 to 15, wherein the phagocytosis deficiency-related disease or condition is:
(a) a musculoskeletal degenerative disease, which is optionally a muscular dystrophy such as Duchenne muscular dystrophy (DMD);
(b) an infectious disease, optionally caused by a bacterium or a virus;
(c) a neurodegenerative disease, which is optionally Alzheimer's disease (AD) or other dementia, Parkinson's disease (PD), Nasu-Hakola disease, prion disease, amyotrophic lateral sclerosis (ALS), Friedreich's ataxia, Huntington's disease, Lewy body disease, spinal muscular atrophy, progressive supranuclear palsy (PSP), or adrenoleukodystrophy (ALD);
(d) inflammation;
(e) an inflammatory disease, which is optionally an autoimmune disease, such as systemic lupus erythematosus (SLE), rheumatoid arthritis (RA), or autoimmune lymphoproliferative syndrome (ALPS); or
(f) a lysosomal storage disease, which is optionally Gaucher disease, Fabry disease, Niemann-Pick disease, Hunter syndrome, Glycogen storage disease II (Pompe disease), or Tay-Sachs disease.

32. The agent or combination of agents for use according to claim 31, wherein the phagocytosis deficiency-related disease or condition is Duchenne muscular dystrophy (DMD).

33. The agent or combination of agents for use according to any one of claims 1 to 32, wherein the subject has phagocytic deficiency.

34. An agent which is cilnidipine or a pharmacologically acceptable salt thereof or a solvate of them for use in a method of treating a subject having pancreatic cancer, optionally wherein the pancreatic cancer is pancreatic ductal adenocarcinoma.

35. The agent for use according to claim 34, wherein the agent is for use as monotherapy.

36. The agent for use according to claim 35, wherein the method comprises administering an amount of the agent to the subject effective to slow growth of a tumor in the subject.

37. The agent for use according to claim 34, wherein the agent is for use in combination with gemcitabine.

38. The agent for use according to claim 37, wherein the method comprises administering an amount of the agent and an amount of the gemcitabine to the subject which together are effective to slow growth of a tumor in the subject.

39. An agent which is cilnidipine or a pharmacologically acceptable salt thereof or a solvate of them for use in a method of treating a subject having Duchenne muscular dystrophy (DMD). The agent for use according to claim 39, wherein the method comprises administering an amount of the agent effect to reduce creatinine kinase (CK) and/or lactose dehydrogenase (LDH) in the subject's plasma.

41. A pharmaceutical composition comprising phagocytes having enhanced phagocytic activity, optionally wherein the phagocytes comprises monocytes, macrophages, neutrophils, dendritic cells, mast cells, or any combination thereof.

42. The pharmaceutical composition of claim 41, which comprises phagocytes that have been contacted with one or more mitochondrial fission inhibitors ex vivo, optionally wherein the one or more mitochondrial fission inhibitors comprise cilnidipine or a pharmacologically acceptable salt thereof or a solvate of them.

43. The pharmaceutical composition of claim 41 or claim 42, for use in a method of treating a subject having a phagocytosis deficiency-related disease or condition, optionally wherein the disease or condition is a cancer.

44. The pharmaceutical composition for use according to claim 43, wherein the phagocytes are autologous to the subject.

45. The pharmaceutical composition for use according to claim 43, wherein the phagocytes are allogeneic to the subject.
